(19) United States
(12) Patent Application Publication
(10) Pub. No.: US 2025/0065319 A1
(43) Pub. Date: Feb. 27, 2025

(54) DEHYDROAROMATIZATION CATALYST AND PREPARATION METHOD THEREOF

(71) Applicant: Korea University Research and Business Foundation, Seoul (KR)

(72) Inventors: Jungkyu CHOI, Seoul (KR); Jaehee SHIM, Seoul (KR); Tristan James SIM, Seoul (KR)

(73) Assignee: Korea University Research and Business Foundation, Seoul (KR)

(21) Appl. No.: 18/811,601

(22) Filed: Aug. 21, 2024

(30) Foreign Application Priority Data
Aug. 21, 2023 (KR) 10-2023-0108975

(51) Int. Cl. B01J 37/10; B01J 29/48; B01J 35/40; B01J 37/02; B01J 37/12

(52) U.S. Cl. CPC B01J 37/10; B01J 29/48; B01J 35/40; B01J 37/0207; B01J 37/0209; B01J 37/12; B01J 2231/00

(57) ABSTRACT

The present invention relates to a dehydroaromatization catalyst and a preparation method thereof, the dehydroaromatization catalyst comprising the steps of: dealuminating zeolite by hydrothermal treatment at a first temperature; and supporting molybdenum (Mo) on the dealuminated zeolite.

### CROSS-REFERENCE TO RELATED APPLICATIONS

This application claims priority to and the benefit of Korean Patent Application No. 10-2023-0108975 filed in the Korean Intellectual Property Office on Aug. 21, 2023, the entire contents of which are incorporated herein by reference.

### TECHNICAL FIELD

The present disclosure relates to a dehydroaromatization catalyst and a preparation method thereof, and more particularly, to a dehydroaromatization catalyst capable of preparing an aromatic compound at a high yield using methane and a method for preparing the same.

### BACKGROUND TECHNOLOGY OF THE INVENTION

Natural gas, the main source of methane (CH₄), is a new alternative energy source to compensate for the depletion of traditional sources such as coal and petroleum. In addition to energy production, methane, which is 70-90% of natural gas, becomes a valuable feedstock for the production of general-purpose chemicals such as methanol (CH₃OH) and acetic acid (CH₃COOH).

In general, in major industrial methods using methane as a supply raw material, high energy is required due to a strong endothermic reaction, and it is practically possible only on a large scale, and CO₂ emission is inevitable. Therefore, the direct conversion pathways of eco-friendly methane, which can be used at small and medium-sized natural gas fields, have been studied for many years.
Korean Patent Laid-Open Publication No. 10-2012-0082889 (2012.07.24)

### CONTENTS OF THE INVENTION

### Summary of the Invention

An object of the present invention is to provide a dehydroaromatization catalyst prepared by a novel preparation method and performing methane dehydroaromatization (MDA) with high efficiency, and a preparation method thereof.

In addition, another object of the present invention is to provide a dehydroaromatization catalyst which can be effectively used in a small-scale reaction device as well as a large-scale reaction device and is environmentally friendly due to a reduction in carbon dioxide emission, and a preparation method thereof.

### Means for Solving Problems

According to an aspect of the present disclosure, embodiments of the present disclosure include a preparation method of a dehydroaromatization catalyst and a dehydroaromatization catalyst. In one embodiment, provided is a preparation method of a dehydroaromatization catalyst, the method comprising: dealuminating a zeolite by hydrothermal treatment at a first temperature; and supporting molybdenum (Mo) in the dealuminated zeolite, wherein a Fourier-transform infrared (FT-IR) spectrum is repeatedly measured a total of 50 to 300 times in the range of 1600-1400 cm⁻¹, and the BrØnsted acid sites obtained by integrating the wavelength of a peak of 1545 cm⁻¹ are 300 μmol·g⁻¹ to 850 μmol·g⁻¹.

In an embodiment, the method may further include primarily calcining the zeolite at a temperature range of about 300° C. to about 700° C. for about 10 hours to about 24 hours before performing the hydrothermal treatment.

In an exemplary embodiment, the first temperature may be 300° C. to 500° C., and the hydrothermal treatment may be performed in a tubular furnace at a flow rate of 800 mL·min⁻¹ to 1500 mL·min⁻¹ of air containing 5 mol % to 20 mol % water vapor for 6 hours to 48 hours.

In an embodiment, the zeolite may have a Si/Al of 10 to 20.

In an embodiment, the content of molybdenum (Mo) may be about 4 wt % to about 8 wt %.

In an exemplary embodiment, the supporting molybdemun (Mo) may include impregnating the dealuminated zeolite in an aqueous solution containing Mo for 10 minutes to 30 minutes at a temperature range of 20° C. to 30° C., drying the dealuminated zeolite at a temperature range of 50° C. to 90° C. for 8 hours to 24 hours, heating the dealuminated zeolite at 0.5° C.·min⁻¹ to 2° C.·min⁻¹. in an air of 200 mL·min⁻¹ to 500 mL·min⁻¹ in order to perform a second calcination for 10 hours to 24 hours at a temperature range of 300° C. to 700° C.

In an embodiment, the Mo-containing aqueous solution may include one or more of ammonium heptamolybdate tetrahydrate ((NH₄)6Mo₇O₂₄·4H₂O), ammonium phosphate hydrate ((NH₄)₃PMo₁₂O₄₀·xH₂O), phosphorus molybdate hydrate (12MoO₃·H₃PO₄·xH₂O), sodium phosphate hydrate (Na₃[P(Mo₃O₁₀)₄]·xH₂O), sodium molybdate 2hydrate (Na₂MoO₄·2H₂O), molybdenum contaminant (MoCl₅), molybdenum(VI) tetrachloride oxide (MoOCl₄), sodium molybdate (Na₂MoO₄), and molybdenum(IV) sulfide (MoS₂).

In an embodiment, the dehydroaromatization catalyst may include soft coke and hard coke, and the hard coke may be 25 parts by weight to 42 parts by weight, the soft coke may be graphite, and the hard coke may be polyaromatic hydrocarbons or polycyclic aromatic compounds based on 100 parts by weight of the soft coke and the hard coke.

In an exemplary embodiment, the dehydroaromatization catalyst may include an internal coke provided inside the pores of the zeolite and an external coke present on the surface of the zeolite, and the external coke may be 60 parts by weight to 75 parts by weight based on total 100 parts by weight of the internal coke and the external coke.

In an embodiment, when the first temperature increases, the amount of BrØnsted acid sites decreases, and the molybdenum may be present in pores of the zeolite.

In an embodiment, the dehydroaromatization catalyst may have an average particle size of 150 μm to 250 μm, and a methane conversion rate according to the following formula may be 2.5% or more.

Methane conversion rate (%)=(number of moles of CH₄ reacted/number of moles of CH₄ supplied)×100 (Formula)

In an embodiment, in the X-ray diffraction (XRD) analysis spectrum using Cu—Kα, the peak corresponding to the (021) plane may not be present, and the peak of (101) may be stronger than the peak of (200/020) and the peak of (111).

In an embodiment, a dehydroaromatization catalyst comprising zeolite and molybdenum (Mo) and prepared according to the above-described method is provided.

In an embodiment, the zeolite may be a ZSM-5-based zeolite, the content of molybdenum (Mo) may be about 4 wt % to about 8 wt %, and the molybdenum may be present in pores of the zeolite.

In an embodiment, the BrØnsted acid sites obtained by repeatedly measuring a Fourier-transform infrared (FT-IR) spectrum a total of 50 to 300 times in a range of 1600-1400 cm⁻¹, and integrating the wavelength of a peak of 1545 cm⁻¹ may be 300 μmol·g⁻¹ to 850 μmol·g⁻¹.

In an embodiment, the dehydroaromatization catalyst may include soft coke and hard coke, and the hard coke may be 25 parts by weight to 42 parts by weight, the soft coke may be graphite, and the hard coke may be polyaromatic hydrocarbons or polycyclic aromatic compounds based on 100 parts by weight of the soft coke and the hard coke.

In an exemplary embodiment, the dehydroaromatization catalyst may include an internal coke provided inside the pores of the zeolite and an external coke present on the surface of the zeolite, and the external coke may be 60 parts by weight to 75 parts by weight based on total 100 parts by weight of the internal coke and the external coke.

In an embodiment, the dehydroaromatization catalyst may have an average particle size of 150 μm to 250 μm, and a methane conversion rate according to the following formula may be 2.5% or more.

Methane conversion rate (%)=(number of moles of CH₄ reacted/number of moles of CH₄ supplied)×100 (Formula)

In an embodiment, in the X-ray diffraction (XRD) analysis spectrum using Cu—Kα, the peak corresponding to the (021) plane may not be present, and the peak of (101) may be stronger than the peak of (200/020) and the peak of (111).

In an embodiment, the dehydroaromatization catalyst may perform a dehydroaromatization reaction with a reactant including methane to prepare an aromatic compound, and the aromatic compound may include any one or more of benzene, toluene, xylene, naphthalene, and coke.

### Effects of the Invention

According to the present invention as described above, it is possible to provide a dehydroaromatization catalyst controlled to have a high reaction rate and efficiency by analyzing the path of a dehydroaromatization reaction of methane, and a preparation method thereof.

In addition, according to the present invention, it is possible to provide a dehydroaromatization catalyst which stably maintains dehydroaromatization performance and has an improved lifespan and an improved cumulative yield of an aromatic compound, and a preparation method thereof.

### BRIEF DESCRIPTION OF THE DRAWINGS

FIG. 1 is a view schematically illustrating a reaction step of a dehydroaromatization catalyst according to an embodiment of the present invention.

FIG. 2 is a flowchart illustrating a preparation method of a dehydroaromatic catalyst according to an embodiment of the present invention.

FIG. 3 is a result of analyzing characteristics of a Mo-supported catalyst (Mo/Z and Mo/Z_x; x=400, 500, 600, 700).

FIG. 4 is a result showing the pore distribution of a Mo-supported catalyst (Mo/Z and Mo/Z_x; x=400, 500, 600, 700).

FIG. 5 shows (a) a Py-adsorbed FT-IR spectrum, (b) a NH₃-TPD profile, and (c) a ²⁷Al MAS NMR spectrum of a Mo-supported catalyst (Mo/Z and Mo/Z_x; x=400, 500, 600, 700).

FIG. 6 shows the peak separation curve of the NH₃-TPD profile of the Mo-supported catalyst (Mo/Z and Mo/Z_x; x=400, 500, 600, 700).

FIG. 7 is a result showing the MDA catalyst performance of a Mo-supported catalyst (Mo/Z and Mo/Z_x; x=400, 500, 600, 700) as a function of Time-on-stream (TOS).

FIG. 8 is a result showing the BT production rate of a Mo-supported catalyst (Mo/Z and Mo/Z_x; x=400, 500, 600, 700) as a function of the reaction time (Time-on-stream, TOS).

FIG. 9 shows the C2 hydrocarbon selectivity as a function of reaction time (TOS) of the Mo-supported catalysts (Mo/Z and Mo/Z_x; x=400, 500, 600, 700).

FIG. 10 shows a ratio of soft coke and hard coke of a Mo-supported catalyst, TGA results, and TG and Ar adsorption analysis results.

FIG. 11 is a BT yield curve of the Mo-supported catalyst according to each reaction time.

FIG. 12 is a profile of the TGA (left axis) and differential mass change (right axis) of the Mo-supported catalyst at reaction times 2, 4, and 6 hours.

FIG. 13 is a profile of the TGA (left axis) and differential mass change (right axis) of the Mo-supported catalyst at reaction times 8, 10, 12 hours.

FIG. 14 is the H—K pore size distribution of the Mo-supported catalyst at reaction times 0, 2, 4, 6 hours.

FIG. 15 is the H—K pore size distribution of the Mo-supported catalyst at reaction times 8, 10, and 12 hours.

FIG. 16 is a graph showing the BAS_{M+S}, BT formation rate, 10-MR pore volume, and BT formation rate corrected with 10-MR pore volume of the Mo-supported catalyst according to each reaction time, respectively.

FIG. 17 is a graph showing the corrected hard coke amount of the Mo-supported catalyst and the ratio of the soft coke ratio to the hard coke according to the reaction time.

FIG. 18 is a graph showing the naphthalene yield of the Mo-supported catalyst according to the reaction time.

FIG. 19 is a schematic diagram of methane dehydrogenation of a Mo-supported catalyst.

FIG. 20 is a result of comparing the performance of the Mo-supported H-ZSM-5 catalyst disclosed in the literature with the performance of the Mo-supported catalysts (Mo/Z, Mo/Z_400) according to the present example.

### DETAILED DESCRIPTION

Details of other embodiments are included in the detailed description and the drawings.

Advantages and features of the present disclosure, and methods of achieving the advantages and features will become apparent with reference to embodiments described below in detail in conjunction with the accompanying drawings. However, the present invention is not limited to the embodiments disclosed below, but may be implemented in various different forms, and unless otherwise specified in the following description, all numbers, values, and/or expressions representing components, reaction conditions, and contents of components in the present invention should be understood to be modified by the term “about” in all cases, as these numbers are essentially approximations reflecting various uncertainties in the measurement occurring in obtaining such values among others. In addition, when a numerical range is disclosed in the present disclosure, the range is continuous, and unless otherwise indicated, the range includes all values from the minimum value of the range to the maximum value including the maximum value. The invention further relates to the inclusion of all integers, including from the minimum value to the maximum value including the maximum value, unless otherwise indicated, when such a range refers to an integer

In addition, in the present disclosure, when a range is described for a variable, it will be understood that the variable includes all values within the described range including the described end points of the range. For example, it will be understood that a range of “5 to 10” includes not only the values of 5, 6, 7, 8, 9, and 10, but also any sub-ranges such as 6 to 10, 7 to 10, 6 to 9, 7 to 9, etc., and also includes any values between integers valid within the scope of the described ranges such as 5.5, 6.5, 7.5, 5.5 to 8.5, 6.5 to 9, etc. For example, it will be understood that a range of “10% to 30%” includes not only values of 10%, 11%, 12%, 13%, etc. and all integers including up to 30%, but also any sub-range of 10% to 15%, 12% to 18%, 20% to 30%, etc., and also includes any value between valid integers within the scope of the range described, such as 10.5%, 15.5%, 25.5%, etc.

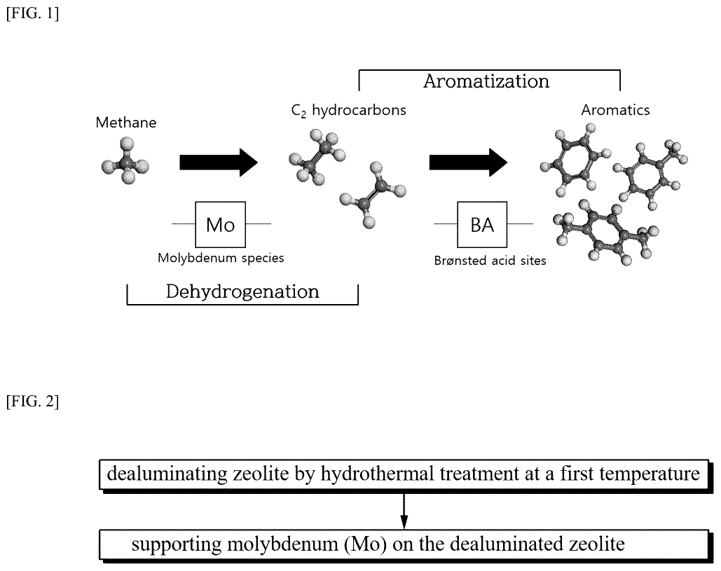

FIG. 1 is a view schematically illustrating a reaction step of a dehydroaromatization catalyst according to an embodiment of the present invention. FIG. 2 is a flowchart illustrating a preparation method of a dehydroaromatic catalyst according to an embodiment of the present invention.

A preparation method of a dehydroaromatization catalyst according to an exemplary embodiment of the present invention includes: dealuminating zeolite by hydrothermal treatment at a first temperature; and supporting molybdenum (Mo) on the dealuminated zeolite. In the dehydroaromatization catalyst prepared by the preparation method of the dehydroaromatization catalyst, after adsorbing Py, the Fourier-transform infrared (FT-IR) spectrum may be repeatedly measured a total of 50 to 300 times in the range of 1600-1400 cm⁻¹, and the BrØnsted acid sites obtained by integrating the wavelength of a peak of 1545 cm⁻¹ may be 300 μmol·g⁻¹ to 850 μmol·g⁻¹.

The dehydroaromatization catalyst may produce lower hydrocarbons including methane, ethane, and the like into aromatic compounds including benzene, toluene, and xylene (BTX), and the like by a methane dehydroaromatization (MDA) reaction. Specifically, the dehydroaromatization catalyst may be used as a catalyst for initial methane conversion in the methane dehydroaromatization reaction by supporting a Mo metal in zeolite, and the desired aromatic compound may be prepared using the produced intermediate material with the BrØnsted acid sites of the zeolite support.

Specifically, the zeolite may include ZSM-5 having pores of 10-membered-ring (10-MR.) among zeolites having various structures. In the Mo-supported ZSM-5 zeolite, the Mo species act as a dehydrogenation catalyst for methane to produce C2 intermediates, while the Bronsted acid sites of ZSM-5 can form aromatics in the zeolite pores. The zeolite may have a Si/Al of about 10 to about 20. When the Si/Al is less than 10, an excessive amount of the Bronsted acid sites of the zeolite may be present to further promote coke formation after reaction, and thus the reaction activity may be deteriorated, and when the Si/Al is greater than 20, sufficient Mo as necessary for reaction may not be supported when Mo is supported.

The dehydroaromatization catalyst may dealuminate the zeolite by hydrothermally treating the zeolite at a first temperature. Subsequently, by supporting Mo in the dealuminated zeolite, the Mo may be fixed to the zeolite.

The dehydroaromatization catalyst may have a BrØnsted acid sites of 300 μmol·g⁻¹ to 850 μmol·g⁻¹, and when the BrØnsted acid sites are less than 300 μmol·g⁻¹, the production efficiency of the aromatic compound is low, which is problematic, and when the BrØnsted acid sites exceed 850 μmol·g⁻¹, the reaction proceeds excessively, so that undesired coke, etc. may be produced, thereby reducing the selectivity of the aromatic compound. The BrØnsted acid sites may be adjusted by removing Al atoms in the framework of the zeolite by hydrothermal treatment of the zeolite.

The preparation method of the dehydroaromatization catalyst may further include primarily calcining the zeolite at a temperature range of 300° C. to 700° C. for 10 hours to 24 hours before performing the hydrothermal treatment. By performing the primary calcination, impurities in the zeolite may be removed to prevent the zeolite structure from collapsing during hydrothermal treatment. In the first calcination, if the temperature is less than 300° C., calcination is not sufficiently performed, and if it exceeds 700° C., the physical properties of the zeolite are changed, which is problematic. In addition, by performing the primary calcination within the above-described time, it is possible to prevent unnecessary process cost increase while sufficiently calcining the zeolite.

The zeolite may be hydrothermally treated at a first temperature after the primary calcination. The first temperature may be 300° C. to 500° C., and the hydrothermal treatment may be performed for 6 hours to 48 hours in a tubular furnace at a flow rate of 800 mL·min⁻¹ to 1500 mL·min⁻¹ of air containing 5 mol % to 20 mol % water vapor. When the first temperature is less than 300° C., dealumination is not sufficiently performed, which is problematic, and when the first temperature is more than 500° C., too few BrØnsted acid sites are formed, which makes it impossible to efficiently produce aromatic compounds.

In the present embodiment, when the first temperature increases, the amount of BrØnsted acid sites may decrease.

In the supporting of molybdenum (Mo), the dealuminated zeolite may be impregnated in an aqueous solution containing Mo at a temperature range of 20° C. to 30° C. for 10 minutes to 30 minutes. Subsequently, the method may include drying the mixture at a temperature range of 50° C. to 90° C. for 8 hours to 24 hours, and then heating the mixture at 0.5° C.·min⁻¹ to 2° C.·min⁻¹ under air of 200 mL·min⁻¹ to 500 mL·min⁻¹ to perform secondary calcining at a temperature range of 300° C. to 700° C. for 10 hours to 24 hours. Specifically, the dealuminated zeolite may be impregnated in the aqueous solution containing Mo at room temperature for 15 minutes.

If the temperature is less than 300° C., it is difficult for the molybdenum to be delivered and fixed in the pores of the zeolite support, and if the temperature is greater than 700° C., the physical properties of the zeolite support may change, which is problematic.

In the secondary calcination, it is possible to remove impurities remaining in the reaction while stably fixing the molybdenum in the support, and when it is less than 300° C., it is difficult to remove impurities after the reaction, and when it is more than 700° C., the molybdenum is formed of an oxide and is provided on the surface of the zeolite support, thereby deteriorating catalyst efficiency.

The content of the molybdenum (Mo) may be 4 wt % to 8 wt %, and the molybdenum may be present in pores of the zeolite. The molybdenum may act as a dehydrogenation catalyst for converting methane, which is a raw material, into the C2 intermediate, and when the content is less than 4 wt %, it is not sufficient to act as a dehydrogenation catalyst for methane, and when the content is more than 8 wt %, it blocks fine pores of zeolite to reduce reaction efficiency. In addition, when the molybdenum is present on the outer surface of the zeolite, the activity thereof is lower than that present therein, which is problematic. Molybdenum present on the outer surface can be identified using TEM or XPS.

The aqueous solution including Mo may include at least one of ammonium heptamolybdate tetrahydrate ((NH₄)6Mo₇O₂₄·4H₂O), ammonium phosphate hydrate ((NH₄)₃PMo₁₂O₄₀·xH₂O), phosphomolybdate hydrate (12MoO₃·H₃PO₄·xH₂O), sodium phosphate hydrate (Na₃[P(Mo₃O₁₀)₄]·xH₂O), sodium molybdate two hydrate (Na₂MoO₄·2H₂O), molybdenum contaminant (MoCl₅), molybdenum(VI) tetrachloride oxide (MoOCl₄), sodium molybdate (Na₂MoO₄), molybdenum(IV) sulfide (MoS₂).

The dehydroaromatization catalyst may comprise soft coke and hard coke. Based on 100 parts by weight of the total weight of the soft coke and the hard coke, the hard coke may be 25 parts by weight to 42 parts by weight. The soft coke may be graphite, and the hard coke may be polyaromatic hydrocarbons or polycyclic aromatic compounds. Specifically, the soft coke may be artificial graphite, and the hard coke may include any one or more of biphenylene, acenaphthylene, phenanthrene, anthracene, fluoranthene, pyrene, xylene, naphthalene, benzo(A)pyrene (BaP), benzo[E]pyrene (BeP), benzo[a]anthracene (BaA), chrysene (CHR), benzo[b]fluoranthene (BbFA), benzo[j]fluoranthene (BjFA), benzo[k]fluoranthene (BkFA), and di-benzo[a,h]anthracene (DBAhA).

When the hard coke is less than 25 parts by weight, the catalytic reaction efficiency is deteriorated, and when the hard coke is more than 42 parts by weight, the fine pores of the zeolite are blocked by the hard coke, thereby deteriorating the reaction efficiency.

In addition, the dehydroaromatization catalyst may comprise an internal coke provided inside the pores of the zeolite, and an external coke present on the surface of the zeolite. Based on 100 parts by weight of the total of the internal coke and the external coke, the external coke may be 60 parts by weight to 75 parts by weight. Specifically, the external coke may be 63 parts by weight to 73 parts by weight. The external coke may be provided within the above-described range, thereby improving the yield of the aromatic compound. Here, the internal coke or the external coke may be determined by a location, for example, the inside or the surface of the pores, present with respect to the zeolite, regardless of the type of the hard coke and the soft coke.

The dehydroaromatization catalyst may have an average particle size of 150 μm to 250 μm, and a methane conversion rate according to the following formula may be 2.5% or more.

Methane conversion rate (%)=(number of moles of CH₄ reacted/number of moles of CH₄ supplied)×100 (Formula)

The methane conversion rate according to the above (Formula) may be measured by supplying a reaction gas containing methane of CH₄ and Ar (CH₄/Ar=9/1, v/v) at a rate of 5 mL·min⁻¹ and fixing the reaction gas with 0.2 g of the dehydroaromatization catalyst to maintain a gas capacity per hour of 1500 mL·g⁻¹·h⁻¹. Specifically, the catalyst is thermally activated by heating the catalyst at 500° C. for 1 hour under a flow of He having a heating rate of 2.5° C.·min−1, and the catalyst is measured by maintaining a temperature of 500° C., and then heating the catalyst to 700° C. with a heating rate of 2.0° C.·min⁻¹. At this time, the initial state of the catalyst starts to be measured 10 minutes after the reaction gas is supplied so that the experiment is performed in a state where the methane completely covers the catalyst.

In the dehydroaromatization catalyst, in the X-ray diffraction (XRD) analysis spectrum using Cu—Kα, the peak corresponding to the (021) plane may not be present, and the peak of (101) may be stronger than the peak of (200/020) and the peak of (111). The peak corresponding to the (021) plane is a representative XRD peak of MoO₃ shown at about 27.3°, and in the dehydroaromatization catalyst according to the present embodiment, an oxide of Mo, for example, MoO3, or the like, is hardly present on the outer surface of the zeolite, and Mo is uniformly dispersed in the zeolite channel. In addition, the peak of (101) was stronger than the peak of (200/020) and the peak of (111) in the dehydroaromatization catalyst, which means that the crystallinity of the zeolite was increased by hydrothermal treatment.

According to another aspect of the present invention, the present invention includes a dehydroaromatization catalyst prepared according to the above-described method.

The dehydroaromatization catalyst uses ZSM-5-based zeolite as a support, and 4 wt % to 8 wt % of molybdenum (Mo) may be uniformly sprayed inside the pores of the zeolite.

In addition, in the dehydroaromatization catalyst according to the present embodiment, the size of the micropores hardly changes even by hydrothermal treatment, and the BrØnsted acid sites obtained by repeatedly measuring the Fourier-transform infrared (FT-IR) spectrum 50 to 300 times in a range of 1600-1400 cm⁻¹, and integrating the wavelength of the peak of 1545 cm⁻¹ may be 300 μmol·g⁻¹ to 850 μmol·g⁻¹. In the dehydroaromatization catalyst, the Mo is involved in dehydrogenating methane to produce a C₂ intermediate product, and the BrØnsted acid site can produce an aromatic compound of the C₂ intermediate product.

The dehydroaromatization catalyst may perform a dehydroaromatization reaction with a reactant including methane to prepare an aromatic compound, and the aromatic compound may include any one or more of benzene, toluene, xylene, naphthalene, and coke.

Hereinafter, Examples and Comparative Examples of the present invention will be described. However, the following Examples are only preferred embodiments of the present invention, and the scope of the present invention is not limited by the following Examples.

### Method for Preparing and Analyzing a Catalyst

### 1. Hydrothermal Treatment of a H-ZSM-5 Support

A commercial NH₄⁺-ZSM-5 zeolite having a Si/Al ratio of 11.5 (Zeolyst International) was calcined to produce a H⁺-ZSM-5 support. At this time, calcining was performed at 550° C. in a furnace at 1° C.·min⁻¹ for 12 hours under 300 mL·min⁻¹ of air. After the calcining was completed, the H⁺-ZSM-5 support was dealuminated by performing a hydrothermal treatment. The hydrothermal treatment was performed at 400, 500, 600, and 700° C. with different temperatures, respectively, but the other conditions were the same.

The hydrothermal treatment was carried out for 24 hours in a tubular furnace consisting of two heating zones under an air stream (ca. 1000 mL·min⁻¹) containing 10 mol % water vapor. At this time, the H⁺-ZSM-5 support (about 1.0 g) was distributed to two quartz boats to be provided before injecting air into the tubular furnace, and distilled water was injected at a fixed ratio of 4 mL·h⁻¹ into the tubular furnace when the temperature reached 400, 500, 600, and 700° C.

### 2. Mo-Supported H-ZSM-5 Zeolite Catalyst Synthesis

The H-ZSM-5 support (Mo/Z) not subjected to hydrothermal treatment through an initial wetting method, and the H-ZSM-5 supports (Mo/Z_400, Mo/Z_500, Mo/Z_600, Mo/Z_700) subjected to hydrothermal treatment at each temperature of 400, 500, 600 and 700° C. were impregnated in an aqueous solution of ammonium heptamolybdate tetrahydrate ((NH₄)6Mo₇O₂₄·4H₂O, 99%, Sigma-Aldrich) to load the same amount with 6 wt % Mo. Then, after drying at 70° C. overnight, all the dried particles were calcined at 550° C. for 12 hours in a heating lamp at 1° C.·min⁻¹. under 300 mL·min⁻¹ of air to prepare a Mo-supported H-ZSM-5 zeolite catalyst (hereinafter, referred to as a Mo-supported catalyst) in the form of particles.

In addition, for comparison, a catalyst having a Mo loading of 3 wt % and 9 wt % and a commercial NH₄⁺-ZSM-5 zeolite (Zeolyst International) having a Si/Al ratio of 25 were prepared using the same method except that hydrothermal treatment was not performed after calcination, a catalyst having a Mo loading of 6 wt % was not hydrothermal treatment, and the rest were prepared using the same method except that hydrothermal treatment was performed on the catalyst having a different Mo content and the rest had a Mo loading of 3 wt % and 9 wt % when the same Si/Al ratio was 11.5.

Here, the H-ZSM-5 support catalyst in which Mo is supported without performing hydrothermal treatment is referred to as “Mo/Z”, and the H-ZSM-5 support catalyst in which hydrothermal treatment is performed and Mo is supported is referred to as “Mo/Z_x”. Here, x represents a hydrothermal treatment temperature (° C.). Table 1 below shows the catalysts prepared in this Example.

TABLE 1

| | | Hydrothermal | Mo supporting |
|---|---|---|---|
| | Si/Al | treatment | amount |
| Division | ratio | temperature (° C.) | (wt %) |
| Mo/Z | 11.5 | N/A | 6 |
| Mo/Z_400 | 11.5 | 400 | 6 |
| Mo/Z_500 | 11.5 | 500 | 6 |
| Mo/Z_600 | 11.5 | 600 | 6 |
| Mo/Z_700 | 11.5 | 700 | 6 |
| Mo(3)/Z | 11.5 | N/A | 3 |
| Mo(9)/Z | 11.5 | N/A | 9 |
| Mo/Z(25) | 25 | N/A | 6 |

### 3. Catalyst Property Analysis

For every 0.02 deg step using a D/MAX-2500V/PC diffractometer (Rigaku, Japan), a powder X-ray diffraction (XRD) pattern was obtained using Cu Kα radiation (40 kV, 100 mA, λ=0.154 nm) at a rate of 0.5 deg min⁻¹.

Argon (Ar) adsorption-desorption isotherms were measured using a ASAP 2020 analyser (Micromeritics, Inc., USA) at 87 K to determine the texture properties of the catalyst. In addition, all samples were pretreated at 350° C. for 12 hours under vacuum before the Ar adsorption-desorption measurement.

The size distributions of the micropores and mesopores of the catalyst were calculated from the adsorption branches using the Horvath-Kawazoe (H-K) and Barrett-Joyner-Halenda (BJH) methods, respectively.

²⁹Si and ²⁷Al MAS NMR (Magic angle spinning nuclear magnetic resonance) spectra were obtained using a AVANCE III HD 500 MHz spectrometer (Bruker, USA) having a magnetic field of 11.7 T. A ²⁹Si MAS NMR spectrum was detected 1024 times at a rotation speed of 5 kHz at 99.3 MHz, and octakis(trimethylsiloxy)silsesquioxzne (Q8M8) was used as a reference at 11.5 ppm. In addition, the ²⁷Al MAS NMR spectrum was detected 4096 times at a rotation speed of 10 kHz at 130.3 MHz, and 1 M AlCl₃ (aq) was used as a reference of 0 ppm.

In order to identify the type of coke produced from the used catalyst and to estimate the amount thereof, thermogravimetric analysis (TGA) was performed using a Q50 thermogravimetric analyser (TA Instruments, USA). Specifically, 10-20 mg of the pretreated sample was heated from room temperature to 900° C. under an air flow of 100 mL·min⁻¹ at a heating rate of 1.0° C.·min⁻¹. TGA measurement and Ar physical adsorption results were used together to estimate the amount of internal coke and external coke present inside and outside the catalyst, respectively. The amount of internal coke was estimated by multiplying the difference in volume of pores of the catalyst before and after the reaction (i.e., the volume of 10 membered-ring (10-MR.) pores, V_{10-MR}), determined by Ar physical adsorption, by the coke density considered (d=1.22 g·cm⁻³). As a result, the amount of external coke was calculated as the total coke content obtained from the TGA measurement minus the amount of internal coke. The coke density used to estimate the amount of internal coke can represent the density of the polyaromatic coke that is normally assumed to be the composition of the internal coke that is produced during the methane dehydroaromatization (MDA) reaction. Since the coke formed inside the pores of the zeolite is generally polyaromatic, the coke density used to calculate the internal coke may indicate the density of the polyaromatic coke.

Energy dispersive X-ray spectroscopy (EDX) analysis was performed using a field emission scanning electron microscope (FE-SEM, SU-70, Hitachi, Japan), and accordingly, the Mo weight composition and Si/Al ratio of the catalyst were analyzed.

The amount of the total acid sites (i.e., the sum of the Bronsted acid sites and the Lewis acid sites) was estimated by pyridine (Py) adsorption and ammonia (NH3) desorption. Py adsorption experiments were performed using a Hg—Cd—Te (MCT) detector and a Fourier-transform infrared (FT-IR) spectrometer (Nicolet IS50, Thermo Fisher Scientific Inc., USA) equipped with a custom in-situ cell with ZnSe window on both sides. About 1020 mg of the sample was converted to a self-supporting wafer and then placed in a cell and heated to 500° C. under vacuum for at least 6 hours and then cooled to 150° C. to record the background spectrum. Then, helium (He) gas (saturated vapor pressure=2.80 kPa and moving diameter=0.55 nm at 25° C.) passing through the bubbler containing liquid Py was injected into the cell for 20 minutes. Subsequently, the over-adsorbed or weakly adsorbed Py was removed from the sample under vacuum for 1 hour before measuring the FT-IR spectrum. To determine the amount of acid sites, the resulting product, excluding the background spectrum from the obtained FT-IR spectrum, was integrated at wavelengths of 1545 and 1450 cm⁻¹ related to the Bronsted and Lewis acid sites, respectively. The amounts of the BrØnsted and Lewis acid sites were calculated according to the following equation.

Amount of acid sites=FT-IR peak area of corresponding region×[cross-sectional area of self-supporting wafer×(weight of wafer)¹]×(extinction coefficient)¹ (Formula)

Here, the extinction coefficients used at 1545 and 1450 cm⁻¹ are 1.13 and 1.28 cm·μmol−1, respectively. The extinction coefficient is a coefficient indicating how strongly the corresponding chemical species absorbs light at a given wavelength to reduce the light, and may also be expressed as an absorption coefficient.

In addition to FT-IR analysis, temperature program desorption of NH₃ (NH₃-TPD) was performed on the Mo-supported catalyst before and after the reaction using a BELCAT (MicrotractBEL Corp., Japan) analyser. The sample (about 50 mg) was preferentially treated to remove previously adsorbed impurities for 1 hour at 500° C. under He flow. After cooling to 50° C., 30 mL·min⁻¹ of a mixed gas containing 5% NH₃ (He balance) was supplied to the sample cell for 1 hour. After sufficient adsorption, the sample was heated to 800° C. at a heating rate of 10° C.·min⁻¹., and an NH₃ desorption curve was obtained under He 30 mL·min⁻¹. The NH₃ desorption curve according to temperature was subdivided into five areas. The two regions with peaks less than 200° C. are known as physically adsorbed NH₃, SiOH groups, and weak acid sites. Therefore, the acid concentration was measured in consideration of only the peak of 200° C. or higher. Two peaks at about 200-400° C. are known to be due to moderate strength and strong acidic sites, and a single peak above 400° C. is due to the Lewis acid sites. In NH₃-TPD, the term “total BAS_{M+S}” was used as the sum of the acid sites of the medium strength (M) and the strong strength (S). The acid site of the medium strength was NH₃ desorbed at 230° C. to 280° C., and the acid site of the strong strength was calculated based on NH₃ desorbed at 310° C. to 390° C.

### 4. Catalyst Activity Test

In order to evaluate the MDA performance of the Mo-supported catalyst, a fixed bed quartz tube reactor having an inner diameter of 6.5 mm and a length of 420 mm was used. All catalysts were made into pellets and then pulverized, and only particle sizes in the range of 150-250 μm were selected. The reaction gas of CH₄ and Ar (CH₄/Ar=9/1, v/v) was supplied at a rate of 5 mL·min⁻¹, and the amount of catalyst was fixed at about 0.2 g to maintain a gas hourly capacity of about 1500 mL·g⁻¹·h⁻¹ of the reaction gas of CH₄ and Ar. Here, 1500 mL·g⁻¹·h⁻¹ means a space velocity calculated by (gas flow rate of 5 ml·min−1)/(catalyst 0.2 g). In order to precisely analyze, gas chromatograph (GC) analysis was performed using Ar as an internal standard. All reactions were carried out at atmospheric pressure.

In a general setting, a selected particulate catalyst was placed in the middle of a quartz tube reactor into which quartz wool was previously inserted. The quartz wool may be provided before the catalyst is put into the quartz tube, thereby preventing the catalyst from passing as it is. The quartz wool was previously placed inside the quartz tube to fix the cotton in the middle, and then a catalyst was placed on it.

Before the catalyst was placed in the quartz tube reactor, zirconia-silicate ceramic beads were placed on the other side of the quartz wool to reduce space. The catalyst was thermally activated by heating at 500° C. for 1 hour under He flow with a heating rate of 2.5° C.·min⁻¹. After maintaining the temperature of 500° C., the catalyst was heated to 700° C. with a heating rate of 2.0° C.·min⁻¹. Upon reaching 700° C., the feed was converted to a reaction gas. The feed means a supplied gas, and He flows until reaching 700° C., and after reaching 700° C., it is converted into CH₄/Ar gas, which is a reaction gas. The reaction state of the catalyst was first measured 10 minutes after supplying the reaction gas so that the experiment was performed with methane covering the entire catalyst. All measurements were performed using an online GC (6500 GC Young Lin Instrument Co., Republic of Korea) for about 12 hours at 45 minute intervals. In the GC, a packed column (HAYESEP DB 100-120 mesh, 30 ft×1/8½×2.0 mm, Agilent, USA) connected to the TCD was used to detect CH₄ and Ar, and major hydrocarbons such as methane (CH₄), ethane (C₂H₆), ethylene (C₂H₄), propane (C₃H₈), benzene (C₆H₆), toluene (C₇H₈), and naphthalene (C₁₀H₈) involved in the MDA reaction were detected using a capillary column (GS-GASPRO, 30 m×0.32 mm, Agilent, USA) connected to the FID. The temperature of the stainless steel discharge line connected in the GC was maintained at 250° C. to prevent strong adsorption and condensation of aromatic hydrocarbons (e.g., naphthalene). Methane conversion, selectivity of gas hydrocarbons and hard coke and soft coke (solid coke), and hydrocarbon yield were calculated using the following equation.

Methane conversion rate (%)=(number of moles of CH₄ reacted/number of moles of CH₄ supplied)×100

Hydrocarbon selectivity (%)=(Number of moles of hydrocarbon having X of carbon atoms in the hydrocarbon/number of moles of CH₄ reacted)×100

Coke selectivity (%)=100-hydrocarbon selectivity (%)

### Evaluation of Catalyst Properties and Performance

### 1. Structure and Tissue Properties of Mo-Supported H-ZSM-5 Zeolite Catalysts

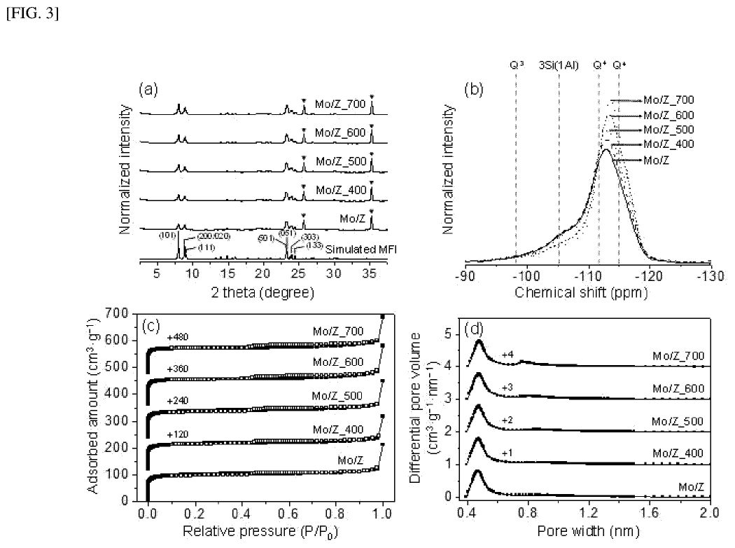

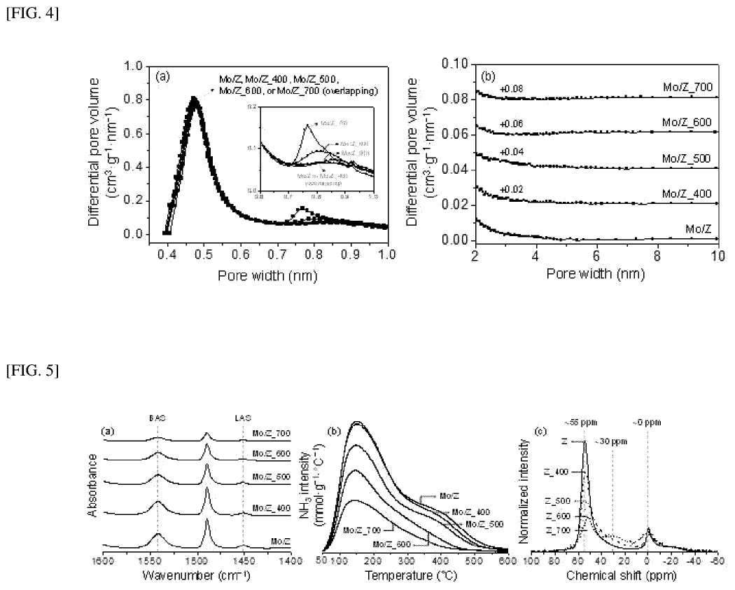

FIG. 3 is a result of analyzing characteristics of a Mo-supported catalyst (Mo/Z and Mo/Z_x; x=400, 500, 600, 700). FIG. 3 shows (a) an XRD pattern, (b) a ²⁹Si MAS NMR spectrum, (c) an Ar isothermal adsorption behavior at 87 K (d) an Ar adsorption behavior with respect to catalysts (Mo/Z) not subjected to hydrothermal treatment and catalysts (Mo/Z_x; x=400, 500, 600, 700° C.) subjected to hydrothermal treatment at temperatures of 400, 500, 600, 700° C. In (a) of FIG. 3, in order to increase the accuracy of comparison, the simulated MFI pattern is included at the bottom, and the inverted triangle shows a peak derived from α-alumina used as an internal standard. FIG. 4 is a result showing the pore distribution of a Mo-supported catalyst (Mo/Z and Mo/Z_x; x=400, 500, 600, 700). FIG. 4 shows the size distribution of (a) micropores and (b) mesopores of the Mo-supported catalyst (Mo/Z and Mo/Z_x; x=400, 500, 600, 700), and shows the H-K pore size distribution and (b) BJH pore size distribution at 0.4-1.0 nm. In the inset of (a), the size distribution of the micropores is an enlarged view of 0.6-1.0 nm.

In (a) of FIG. 3, in the Mo-supported H-ZSM-5 zeolite catalyst (Mo/Z and Mo/Z_x; x=400, 500, 600 and 700), the XRD pattern was similar regardless of the presence or absence of hydrothermal treatment, which means that hydrothermal treatment did not significantly reduce the crystallinity of the zeolite. In addition, it was confirmed that the crystal structure of zeolite was maintained without change even at a temperature of the hydrothermal treatment of 700° C. In addition, no representative XRD peak of MoO₃ was found at ca. 27.3° corresponding to the (021) plane, which means that molybdenum is well dispersed in the zeolite channel and is hardly present on the outer surface and thus is not observed by XRD.

The peak of (101) in the hydrothermally treated catalyst (Mo/Z_x; x=400, 500, 600, 700) was stronger than the peak of (111) in ca. 8° (200/020) and ca. 9° (111) than in the catalyst (Mo/Z) which was not hydrothermally treated. Table 2 below shows the relative crystallinity of the catalyst, and it was confirmed using the combined area of the normalized signal region of the (101) plane at ca. 8° and the normalized signal region of the (200/020) plane and the (111) plane at ca. 9°.

TABLE 2

| Division | Relative crystallinity (%) |
|---|---|
| Mo/Z | 100 |
| Mo/Z_400 | 103 |
| Mo/Z_500 | 122 |
| Mo/Z_600 | 142 |
| Mo/Z_700 | 169 |

(Determined using the combined area of the normalized signal region of the ca. 8° plane and the normalized signal region of the (200/020) and (111) planes at ca. 9°)

Referring to Table 2, it was confirmed that the crystallinity of the catalyst subjected to hydrothermal treatment was higher than that of the catalyst not subjected to hydrothermal treatment, and the crystallinity increased as the temperature of hydrothermal treatment increased. The relative crystallinity refers to an increase in crystallinity of zeolite based on Mo/Z, which is a catalyst that is not subjected to hydrothermal treatment, and the increase in relative crystallinity means that Al is removed from the crystal lattice of zeolite and simultaneously crystallinity is improved. That is, it could be confirmed that as the temperature of the hydrothermal treatment increased, the crystallinity was improved similar to that of the zeolite analog containing a high content of silica.

Referring to (b) of FIG. 3, the ²⁹Si MAS NMR spectrum of each catalyst showed a trend in which the intensity of the Si(0Al) peak (Q⁴) allocated to the tetrahedral Si—O bond (Si—O—Si) of zeolite consistently increased as the temperature of hydrothermal treatment increased. It was confirmed that the strength of Mo/Z was the lowest, the strength gradually increased according to the water heat treatment temperature, and Mo/Z_700 had the largest strength. That is, it was confirmed that the crystallinity of the zeolite was improved by hydrothermal treatment. In addition, the ²⁹Si MAS NMR spectrum of Mo/Z_700 showed that the Q⁴ peak slightly increased compared to that of Mo/Z. This means that the Si—O binding angle related to the monoclinic/orthorhombic transition of ZSM-5 is changed. It was confirmed that the transition was a orthorhombic ZSM-5 structure, which is a partial feature shown at about 0.77 nm in the H-K micropore size distribution of Mo/Z_700 in FIG. 4.

Table 3 shows the textural characteristics of Mo-supported catalysts (Mo/Z and Mo/Z_x; x=400, 500, 600, 700). All the textural characteristics in Table 3 were confirmed from Ar physisorption isotherms at 87 K. Table 4 shows the Mo content and Si/Al ratio (Mo/Z and Mo/Z_x; x=400, 500, 600, 700) of the Mo-supported catalyst.

In addition, the structural strength of the Mo-supported catalyst was confirmed through the tissue characteristics obtained from the results of Ar physical adsorption. The Ar adsorption of all Mo-supported catalysts shows Type I isotherms ((c) of FIG. 3), which is a general form that appears in microporous materials. In addition, it was found that there was no significant difference in the H-K micropore size distribution ((d) of FIG. 3) regardless of whether or not hydrothermal treatment was performed. In addition, the BJH pore size distribution diagram ((b) of FIG. 4) did not show the generation of mesopore structures in the catalyst. Therefore, the BET surface area and total external mesopore surface area (Sₑₓ₊ₘₑₛₒ), micropore volume (V_{micro}) and 10-MR pore volume (V_{10-MR.}) of all Mo-supported catalysts were similar (Table 3). In addition, as a result of the SEM-EDX analysis, it was confirmed that there was no significant change in the characteristics of the catalyst due to Mo support regardless of whether or not hydrothermal treatment was performed. Here, the BJH pore size distribution shows the distribution and presence of mesopores (medium-sized pores with 2-10 nm). (b) of FIG. 4 shows that the mesopores are not formed even after the dealumination treatment, and when the mesopores are formed, a large peak is formed as shown in (a) of FIG. 4.

TABLE 3

| | S_{BET}^{a} | Sₑₓ₊ₘₑₛₒ^{b} | V_{micro}^{c} | V_{10-MR}^{d} |
|---|---|---|---|---|
| Division | (m²/g) | (m²/g) | (m²/g) | (m²/g) |
| Mo/Z | 347 | 53 | 0.13 | 0.076 |
| Mo/Z_400 | 333 | 49 | 0.12 | 0.076 |
| Mo/Z_500 | 331 | 49 | 0.12 | 0.076 |
| Mo/Z_600 | 334 | 31 | 0.12 | 0.075 |
| Mo/Z_700 | 331 | 27 | 0.12 | 0.074 |

^{a}Brunauer-Emmett-Teller (BET) equation
^{b}t-plot equation
^{c}Horvath-Kawazoe (H-K) equation
^{d}Obtained convoluting the micropore size distribution between the 0.4-0.6 nm

TABLE 4

| Division | Mo wt % | Si/Al ratio |
|---|---|---|
| Mo/Z | 5.0 ± 0.3 | 10.1 ± 0.1 |
| Mo/Z_400 | 5.3 ± 0.3 | 10.3 ± 0.2 |
| Mo/Z_500 | 5.2 ± 0.3 | 10.4 ± 0.3 |
| Mo/Z_600 | 4.8 ± 0.2 | 10.0 ± 0.2 |
| Mo/Z_700 | 4.9 ± 0.4 | 10.2 ± 0.2 |

(Obtained through SEM-EDX analysis in various mapping regions of each catalyst for statistical measurement)

### 2. Acid Site Characteristics of a Mo-Supported Catalyst

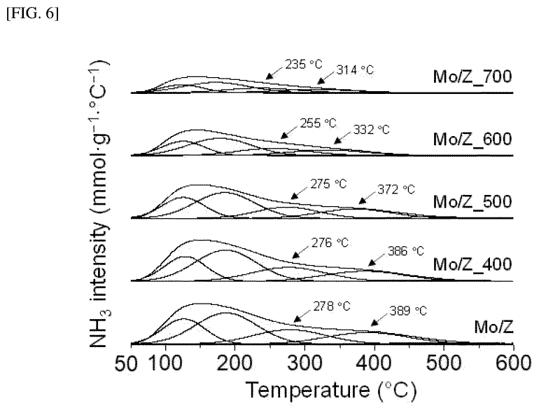

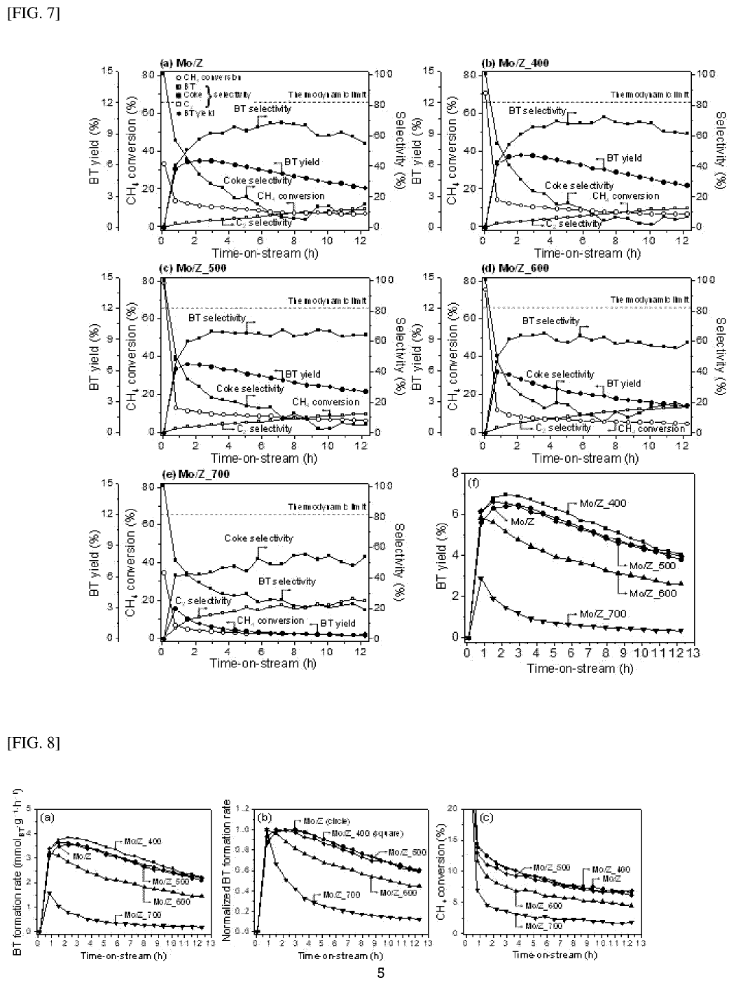

FIG. 5 shows (a) a Py-adsorbed FT-IR spectrum, (b) a NH₃-TPD profile, and (c) a H⁺-ZSM-5 support (Z) not impregnated with Mo and a ²⁷Al MAS NMR spectrum of hydrothermally treated H⁺-ZSM-5 (Z_x; x=400, 500, 600, 700) of a Mo-supported catalyst (Mo/Z and Mo/Z_x; x=400, 500, 600, 700). In (a) of FIG. 5, BAS and LAS refer to the BrØnsted and Lewis acid sites, respectively, and in (c) of FIG. 5, they are ²⁷Al MAS NMR spectra of the H-ZSM-5 support (Z), which has not been hydrothermally treated and on which Mo is not supported, and the support (Z_x; x=400, 500, 600, 700), which has been hydrothermally treated at each temperature. FIG. 6 shows the peak separation curve of the NH₃-TPD profile of the Mo-supported catalyst (Mo/Z and Mo/Z_x; x=400, 500, 600, 700). In FIG. 6, the peaks indicated by arrows represent BrØnsted acid sites of medium strength (200 to 300° C.) and strong strength (300 to 400° C.), respectively. For convenience, the NH₃ desorption temperature of the acidic region was indicated.

Table 5 shows the contents of the BrØnsted and Lewis acid sites of the Mo-supported catalysts (Mo/Z and Mo/Z_x; x=400, 500, 600, 700).

TABLE 5

| | Based on Py-adsorbed FT-IR^{a} | | | Based on NH₃-TPD | | |
|---|---|---|---|---|---|---|
| | Total acid | Brønsted | Lewis acid | Total acid | Brønsted | Lewis acid |
| | sites | acid sites^{b} | sites^{e} | sites | acid sites^{b} | sites^{e} |
| Division | (μmol/g) | (μmol/g) | (μmol/g) | (μmol/g) | (μmol/g) | (μmol/g) |
| Mo/Z | 947.8 | 856.7 | 91.1 | 615.5 | 579.5 | 36 |
| Mo/Z_400 | 883.6 | 808.3 | 75.3 | 565.4 | 538.8 | 26.6 |
| Mo/Z_500 | 524.5 | 478.5 | 46 | 427 | 405.5 | 21.5 |
| Mo/Z_600 | 382.9 | 348.5 | 34.4 | 295.6 | 283.8 | 11.8 |
| Mo/Z_700 | 196.5 | 173.9 | 22.6 | 193.9 | 184.6 | 9.3 |

^{a}Determine after vacuuming crystals at 150° C.
^{b}Estimated by integrating the FT-IR spectrum of the Py-adsorbed sample at 1545 cm⁻¹
^{c} Estimated by integrating the FT-IR spectrum of the Py-adsorbed sample at 1545 cm⁻¹
^{d} Estimated by integrating and adding two NH₃ desorption peaks at 200-400° C. (intermediate acid + strong acid) (where intermediate acid is measured at 200-300° C., strong acid at 300° C.)
^{e}Estimation by integrating the NH₃ desorption peak at a temperature above 400° C.

The acid concentrations of all Mo-supported catalysts determined by Py-adsorption FT-IR and NH₃-TPD analysis are shown in (a) and (b) of FIG. 5 and are summarized in Table 3. As the temperature of the hydrothermal treatment increased, the total amount of BAS (BrØnsted acid sites) identified in the Py-adsorption FT-IR and NH₃-TPD analyzes decreased. That is, it was confirmed that the total amount of BAS gradually decreased as the temperature of the hydrothermal treatment increased. In addition, in order to classify the intensity based on the NH₃ desorption temperature along with the amount of acid sites, in particular, NH₃-TPD analysis was used together. In FIG. 6, the NH₃-TPD profiles are shown separately.

When the H-ZSM-5 support was hydrothermally treated, the total BAS_{M+S} (medium BAS and strong BAS) was greatly reduced compared to the Mo/Z sample that was not hydrothermally treated. Compared to Mo/Z, Mo/Z_400 showed a slight decrease in the amount of BAS_{M+S} (579.5 μmol·g⁻¹ vs. 538.8 μmol·g−1 of Mo/Z), the NH3 desorption temperature was almost similar, and it was confirmed that the desorption intensity was similar (386° C. and 276° C. vs. 389° C. and 278° C. of Mo/Z). When the temperature of the hydrothermal treatment was further increased to 500, 600, and 700° C., the concentration and strength of the acid were greatly reduced. That is, the amount and strength of BAS_{M+S} can be controlled by dealumination in the framework of zeolite by hydrothermal treatment, and the strength decreases as the temperature of the hydrothermal treatment increases.

Since BAS is closely related to Al atoms in the framework of tetrahedral zeolite, the status of Al species contained in the untreated H-ZSM-5 support and the hydrothermally treated H-ZSM-5 support (Z and Z_x; x=400, 500, 600, 700) at each temperature was analyzed by ²⁷Al MAS NMR spectroscopy before loading Mo. Referring to (c) of FIG. 5, in all H-ZSM-5 supports not including Mo, a framework Al peak organized into tetrahedrons at about 55 ppm appeared. In addition, when the temperature of the hydrothermal treatment increased, the intensity of the peak gradually decreased, which corresponded to the decrease in the amount of BAS and the decrease in the monotonicity of the intensity ((a) and (b) of FIG. 2 and FIG. 6). Table 6 shows the peak area around 55 ppm analyzed by ²⁷Al MAS NMR spectroscopy.

TABLE 6

| | Area of tetrahedral | Reduced ratio (%) |
|---|---|---|
| Division | (55 ppm) peak | based on the area of Z |
| Z | 50647794.51 | n/a |
| Z_400 | 46352068.54 | 8.481565709 |
| Z_500 | 30470749.63 | 39.83795361 |
| Z_600 | 21412378.82 | 57.72297879 |
| Z_700 | 16991185.16 | 66.45227037 |

Referring to Table 6, it was confirmed that as the temperature of the hydrothermal treatment increased, the peak area around 55 ppm gradually decreased. That is, as shown in the results of the ²⁷Al MAS NMR spectrum, it was confirmed that Al atoms were effectively extracted from the zeolite framework by the hydrothermal treatment according to the present embodiment. The Al peak near 0 ppm representing the Al species present outside of the zeolite framework (outer framework Al species) was found in all kinds of supports. The lost amount of Al species in the framework of all hydrothermally treated H-ZSM-5 supports did not quantitatively correspond to the increase in NMR peaks corresponding to the outer framework Al species.

Z, which is a H-ZSM-5 support not subjected to hydrothermal treatment, and Z_400, which is a H-ZSM-5 support subjected to hydrothermal treatment at 400° C., were compared. For Z_400, a broad peak was observed near 30 ppm. Abroad peak near 30 ppm was mainly shown in Al having a different pentagonal structure or Al of a distorted tetrahedron, and a broad peak near 30 ppm was shown to be stronger as the temperature of hydrothermal treatment increased (Z_500<Z_600<Z_700). In addition, the upward shift of the peak at a high hydrothermal treatment temperature (x≥500° C.) also means a framework Al species organized into a tetrahedron.

Dealumination can be confirmed by quantifying the spectral intensity of the Al species as a whole in the zeolite support, but there were some differences by the framework Al species that were not measured or not organized into tetrahedrons. On the other hand, when hydrothermal treatment was performed at a high temperature, the monotone reduction of the tetrahedral framework Al species ((c) of FIG. 5) was consistent with the same trend in the amount and intensity of BAS described above.

### 2. Evaluation of MDA Catalyst Performance of a Mo-Supported Catalyst

FIG. 7 is a result showing the MDA catalyst performance of a Mo-supported catalyst (Mo/Z and Mo/Z_x; x=400, 500, 600, 700) as a function of Time-on-stream (TOS). In FIG. 7, (a) Mo/Z, (b) Mo/Z_400, (c) Mo/Z_500, (d) Mo/Z_600, and (e) Mo/Z_700 show MDA catalyst performance over time, and (f) their BT yield. In addition, in (a)-(e), the left axis represents CH₄ conversion and BT yield, and the right axis represents selectivity of BT, C₂ and coke. In addition, the dashed line shows the thermodynamic limit of the conversion of CH₄ to benzene at 700° C. All MDA reactions were carried out at 700° C. for about 12 hours at atmospheric pressure, and the feed rate of the reaction gas was maintained at 5 mL·min⁻¹ (CH₄/Ar=9/1 volume ratio).

In (f) of FIG. 7, the yield of benzene+toluene (BT) was separately represented as a function of TOS, and the BT yield in TOS up to about 12 hours was shown in the order of increasing activity of the catalyst (Mo/Z_400>Mo/Z≈Mo/Z_500>Mo/Z_600>Mo/Z_700). In addition, Mo/Z_400, which has a lower BAS amount than Mo/Z, was better than Mo/Z in MDA catalyst performance. On the other hand, Mo/Z_x at which the hydrothermal treatment temperature exceeded 400° C.; x=500, 600 and 700 showed relatively reduced catalyst performance. All Mo-supported catalysts began to produce BT products after a TOS of approximately 1 hour, followed by a catalyst inert step where the BT yield decreased. The BT yield trends over time for the Mo-supported catalyst were all similar (achieving maximum (max) BT yield in the initial reaction stage before the catalyst inert step), but at different TOS the maximum BT yield was achieved.

Mo/Z and Mo/Z_400 showed the highest BT yield at similar TOS (yield of about 6.4% at Mo/Z., yield of about 7.0% at Mo/Z_400.). Although Mo/Z and Mo/Z_500 had similar catalytic activity (FIG. 7f), Mo/Z_500 achieved a maximum BT yield (about 6.6%) at faster TOS (about 1.5 hours), while Mo/Z achieved a maximum BT yield (about 6.4%) at later TOS (about 2.2 hours). This means that Mo/Z and Mo/Z_500 show different BT generation tendencies. At Mo/Z_600, the tendency to produce BT differed significantly, with a maximum BT yield (about 5.8%) achieved at TOS about 1 hour earlier. Mo/Z_700, which was subjected to hydrothermal treatment at the highest temperature of 700° C., showed very low performance (maximum BT yield is about 2.9%). That is, it was confirmed that the effect of hydrothermal treatment affects the BT yield generated according to TOS.

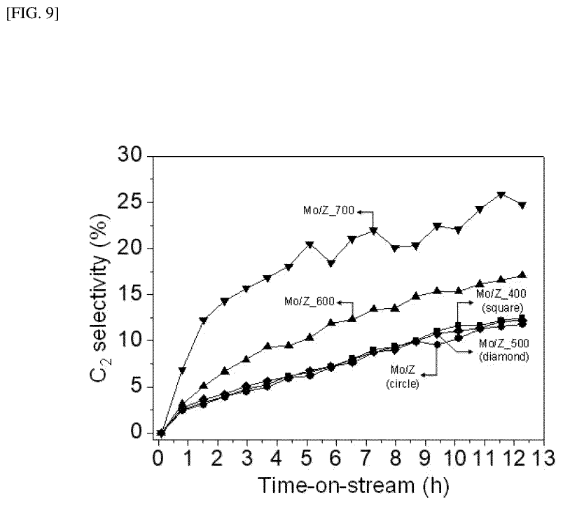

FIG. 8 is a result showing the BT production rate of a Mo-supported catalyst (Mo/Z and Mo/Z_x; x=400, 500, 600, 700) as a function of the reaction time (Time-on-stream, TOS). In FIG. 8, changes in TOS were shown according to (a) BT production rate, (b) normalized BT production rate, and (c) CH₄ conversion of Mo-supported catalyst (Mo/Z and Mo/Z_x; x=400, 500, 600 and 700). Specifically, in (a), the BT yield was multiplied by the feed flow rate of methane to obtain the production rate, and in (b), the production rate was normalized using the maximum BT production rate achieved by each catalyst. FIG. 9 shows the C2 hydrocarbon selectivity as a function of reaction time (TOS) of Mo-supported catalysts (Mo/Z and Mo/Z_x; x=400, 500, 600, 700).

In order to confirm the MDA catalyst performance of the Mo-supported catalyst, the BT production rate was confirmed ((a) of FIG. 8). Here, the BT production rate was calculated by multiplying the BT yield by the supply flow rate of methane. In addition, the maximum BT production rate was used to normalize all other production rates ((b) of FIG. 8).

Referring to (b) of FIG. 8, despite the difference between the absolute values of the BT generation rates of Mo/Z and Mo/Z_400, the BT generation behavior according to TOS was similar. In addition, as described above, Mo/Z_500 began to decrease due to catalyst deactivation after achieving the maximum rate of BT production at a faster TOS of about 1.5 hours.

Mo/Z_500 showed a similar trend to CH₄ conversion of Mo/Z and Mo/Z_400 ((c) of FIG. 8), and thus the degree and form of dispersion of Mo in Mo/Z_500 is expected to be similar to those of Mo/Z and Mo/Z_400. On the other hand, the only difference that Mo/Z_500 has for Mo/Z, Mo/Z_400 lies in the spatial interaction between Mo species and BAS. The spatial interaction of Mo/Z_500 is thought to be due to the presence of other Al species that have occurred in the zeolite support. Specifically, in (c) of FIG. 8, as the hydrothermal treatment temperature increases, an Al peak generated at 30 ppm is observed, and it is determined that Mo/Z_500 has a new Al formation to change the interaction between Mo and BAS, and as a result, shows a trend different from that of Mo/Z and Mo/Z_400.

On the other hand, both Mo/Z_600 and Mo/Z_700 achieved a maximum BT yield at a TOS of about 1 hour. Similar to Mo/Z_500, it is predicted that Mo dispersed in Mo/Z_600 and Mo/Z_700 was affected by the framework Al species rather than the tetrahedron produced by hydrothermal treatment ((c) of FIG. 5). That is, it was determined that the interaction between Mo and BAS was affected by new Al ((c) of FIG. 5, Al peak shown at 30 ppm) generated in the hydrothermally treated zeolite. Mo/Z_600 and Mo/Z_700 decreased the rate of BT production after achieving maximum BT yield. This is because both catalysts of Mo/Z_600 and Mo/Z_700 had low CH4 conversion.

Referring to (c) of FIG. 8, Mo/Z_600 and Mo/Z_700 exhibited a lower CH4 conversion rate not only in the initial stage (about 1 hour) but also in the remaining portion of the reaction time compared to other catalysts. This is believed to be due to the excessive dealumination of the support of Mo/Z_600 and Mo/Z_700, which removed most of the Al species from the tetrahedral framework that immobilized the Mo species.

In the Mo-supported catalyst, the outer Mo species located on the outer surface are less active than the Mo species present inside the zeolite pores. That is, it is judged that Mo/Z_600 and Mo/Z_700 are excessively dealuminated and thus most of the Mo species exist on the outer surface, and as a result, both CH4 conversion and BT yield are low.

Referring to FIG. 9, C2 selectivity of Mo/Z_600 and Mo/Z_700 was higher than that of Mo/Z_400 and Mo/Z. This means that Mo/Z_600 and Mo/Z_700 were selective for C2 hydrocarbons, but these intermediate products were not further aromatized due to the lack of BAS. That is, it was confirmed that when the zeolite was subjected to hydrothermal treatment at 600° C. and 700° C., Al was removed in excess from the zeolite framework, and as a result, most of the Mo species were distributed on the outer surface, thereby reducing the catalytic activity and significantly reducing the BAS concentration in the zeolite support.

3. Coke Analysis on Mo-Supported Catalysts after Reaction

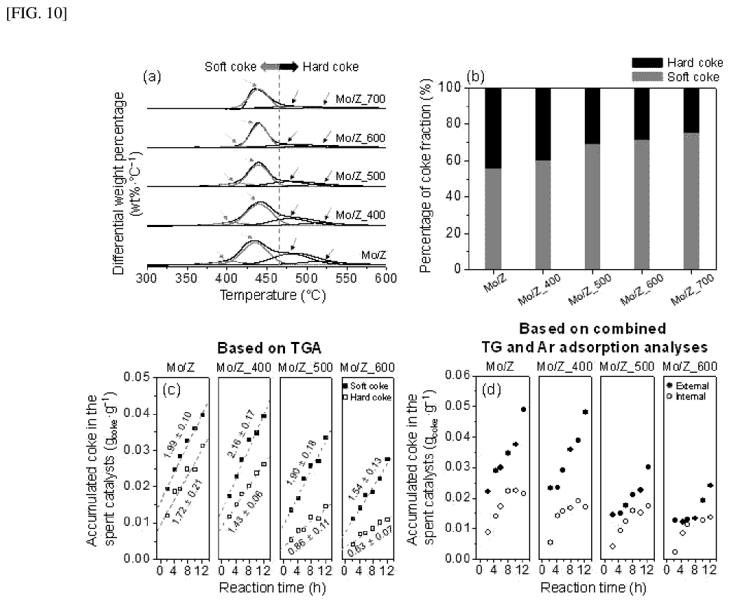

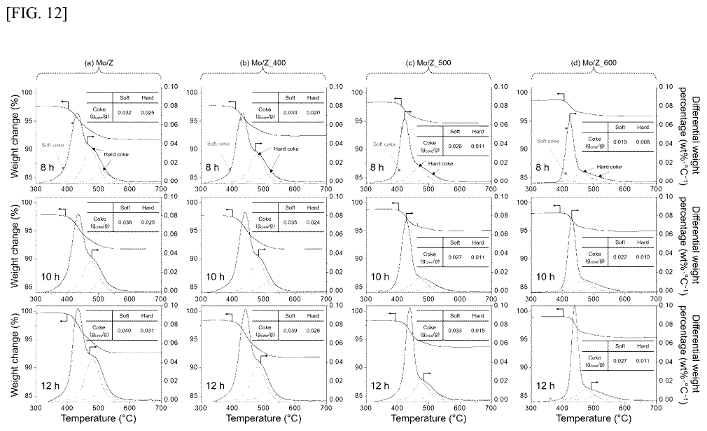

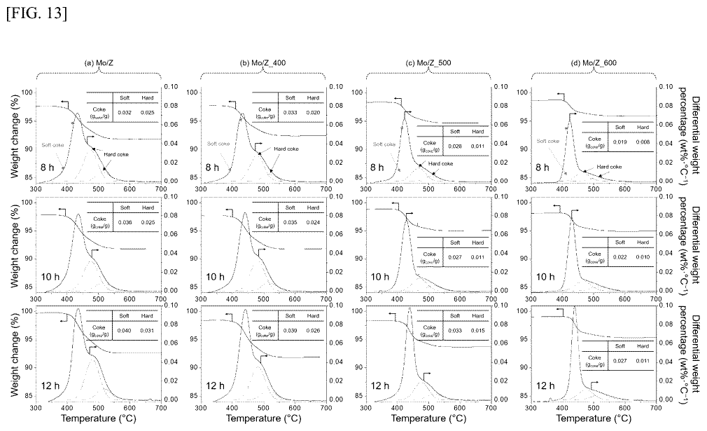

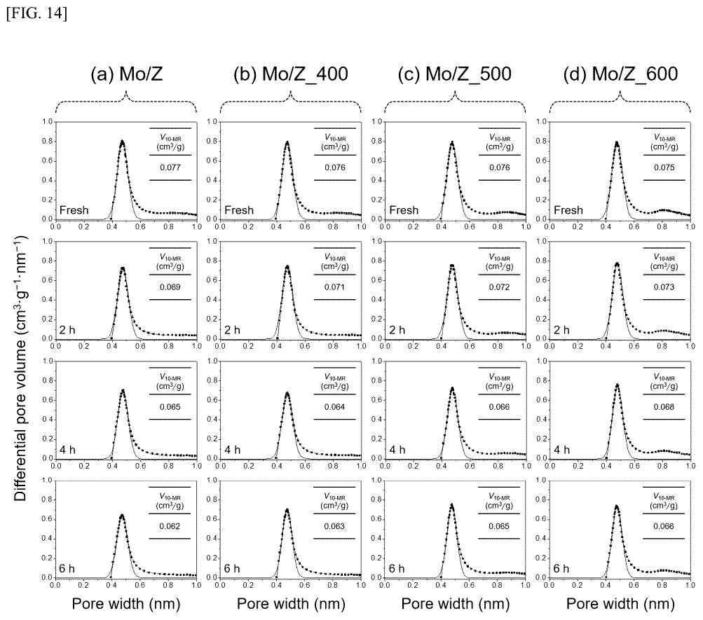

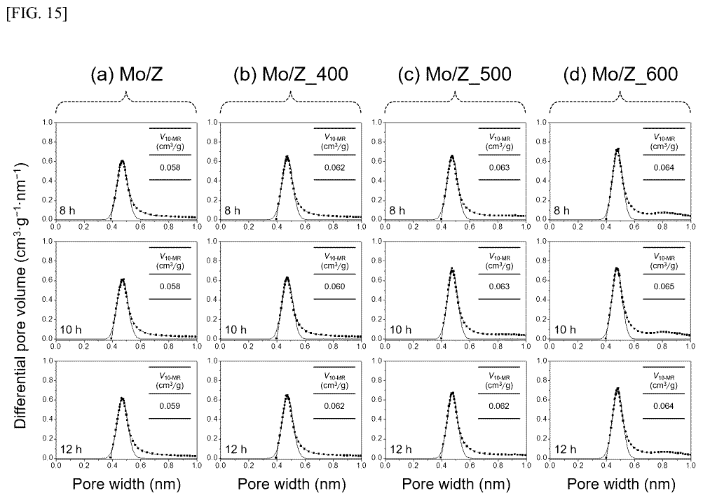

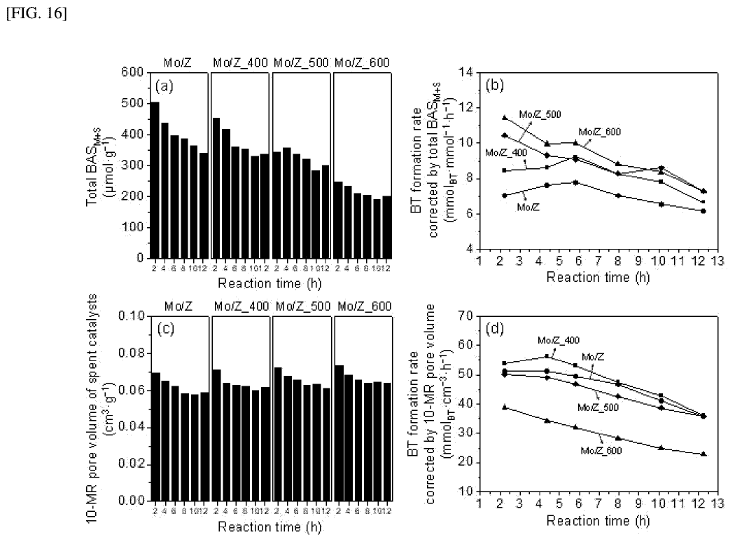

FIG. 10 shows a ratio of soft coke and hard coke of a Mo-supported catalyst, TGA results, and TG and Ar adsorption analysis results. (a) of FIG. 10 is a differential weight percentage profile (TGA measurement result) of the Mo-supported catalyst after 12 hours of reaction. Here, curves indicated by gray and black arrows represent soft coke and hard coke, respectively. (b) of FIG. 10 shows the percentage of soft coke and hard coke formed in the Mo-supported catalyst after 12 hours of reaction. (c) and (d) of FIG. 10 show cumulative amounts of soft coke and hard coke of a Mo-supported catalyst (Mo/Z and Mo/Z_x; x=400, 500, and 600) according to reaction time. Here, (c) of FIG. 10 is a TGA measurement result, and (d) is determined by TG and Ar adsorption analysis results. (c) of FIG. 10 shows the results of confirming the amounts of soft coke and hard coke in the catalyst used for the reaction for two representative peaks at a low temperature (about 440° C.) and a high temperature (about 480° C.) of the TGA profile. FIG. 12 is a profile of the TGA (left axis) and differential mass change (right axis) of the Mo-supported catalyst at reaction times 2, 4, and 6 hours. FIG. 13 is a profile of the TGA (left axis) and differential mass change (right axis) of the Mo-supported catalyst at reaction times 8, 10, 12 hours. In FIGS. 12 and 13, (a) Mo/Z, (b) Mo/Z_400, (c) Mo/Z_500 and (d) Mo/Z_600 are shown for about 2, 4, 6, 8, 10 and 12 hours, respectively, and the approximate reaction time of the spent catalyst is indicated in the lower left corner. FIG. 14 is the H-K pore size distribution of the Mo-supported catalyst at reaction times 0, 2, 4, 6 hours. FIG. 15 is the H-K pore size distribution of the Mo-supported catalyst at reaction times 8, 10, and 12 hours. In FIGS. 14 and 15, (a) Mo/Z, (b) Mo/Z_400, (c) Mo/Z_500, and (d) Mo/Z_600. The approximate reaction time of each spent catalyst is shown in the lower left corner. FIG. 16 is a graph showing the BAS_{M+S}, BT formation rate, 10-MR pore volume, and BT formation rate corrected with 10-MR pore volume of the Mo-supported catalyst according to each reaction time, respectively. In FIG. 16, (a) is the result of confirming the total available BASM+S determined by NH₃-TPD after recovering the reacted Mo-supported catalyst (Mo_Z and Mo/Z_x; x=400, 500, and 600) after each reaction time (about 2, 4, 6, 8, 10, and 12 h), and (b) is the corrected BT formation rate with the total available BAS_{M+S} obtained in (a). (c) of FIG. 16 shows the 10-MR pore volume of the catalyst recovered after each reaction time (about 2, 4, 6, 8, 10, and 12 h) (a value calculated by decomposing the H-K pore size distribution shown in FIGS. 14 and 15), and (d) shows the BT formation rate corrected by the 10-MR pore volume obtained in (c).

Table 7 is a linear fit equation for each of the soft coke and the hard coke. In Table 7, numerical values (i.e., the slope of the fit equation) are described to show the estimated accumulation rate (mg_{coke}·g⁻¹·h⁻¹) for each soft coke and hard coke. Table 8 shows the ratio of soft coke to hard coke.

TABLE 7

| Division | Soft coke | Hard coke |
|---|---|---|
| Mo/Z | y = (0.00199 ± 0.00010)t + | y = (0.00172 ± 0.00021)t + |
| | (0.01574 ± 0.00081) | (0.00938 ± 0.0017) |
| Mo/Z_400 | y = (0.00216 ± 0.00017)t + | y = (0.00143 ± 0.00006)t + |
| | (0.01356 ± 0.00131) | (0.00885 ± 0.00044) |
| Mo/Z_500 | y = (0.00190 ± 0.00018)t + | y = (0.00086 ± 0.00011)t + |
| | (0.00950 ± 0.00139) | (0.00359 ± 0.00085) |
| Mo/Z_600 | y = (0.00154 ± 0.00013)t + | y = (0.00063 ± 0.00007)t + |
| | (0.00747 ± 0.00105) | (0.00339 ± 0.00055) |

TABLE 8

| Division | Soft coke (%) | Hard coke (%) |
|---|---|---|
| Mo/Z | 55.97 | 44.03 |
| Mo/Z_400 | 60.2 | 39.8 |
| Mo/Z_500 | 69.67 | 30.33 |
| Mo/Z_600 | 71.9 | 28.1 |
| Mo/Z_700 | 75.96 | 24.04 |

The Mo-supported catalyst according to this example was subjected to hydrothermal treatment to dealuminate the zeolite support in order to increase the BT intermediate product before the hard coke was produced. In addition, the hard coke reduces the stability of the catalyst, and the content of the hard coke is reduced by the dealumination, thereby making it possible to improve the stability of the catalyst. In order to confirm this, the Mo-supported catalyst was recovered according to each reaction time (about 2, 4, 6, 8, 10 and 12 hours), and soft coke and hard coke were analyzed for this.

Specifically, soft coke and hard coke were analyzed using two approaches: (1) a method using only TGA measurements and (2) a method combining TG and Ar adsorption analysis. The type of coke in the catalyst used was confirmed using the TGA measurement.

In the differential weight percentage profile of the Mo-supported catalyst recovered after 12 hours of reaction ((a) of FIG. 10), two major weight reductions at about 440° C. and 480° C. were distinguished, and two peaks for soft coke and hard coke, respectively, were shown. The weight reduction at low temperatures (about 440° C.) corresponds to coke (known as soft coke) similar to graphene by decomposition of the intermediate C2 hydrocarbons, and the weight reduction at high temperatures (about 480° C.) corresponds to coke (known as hard coke) similar to aromatics by polymerization of the aromatic products.

The coke whose mass is reduced at a low temperature of about 440° C. is a soft coke generated by the decomposition of intermediate C2 hydrocarbons, and the coke whose mass is reduced at a high temperature of about 480° C. is a hard coke formed by the polycondensation of aromatic substances. It was confirmed that Mo/Z_x subjected to the hydrothermal treatment had a lower ratio of hard coke than Mo/Z. In addition, it could be confirmed that as the temperature of the hydrothermal treatment increased, the ratio of hard coke decreased compared to the ratio of soft coke. That is, it can be seen that the higher the temperature in the process of dealumination, the lower the BrØnsted acid site which promotes the formation of a hard coke.

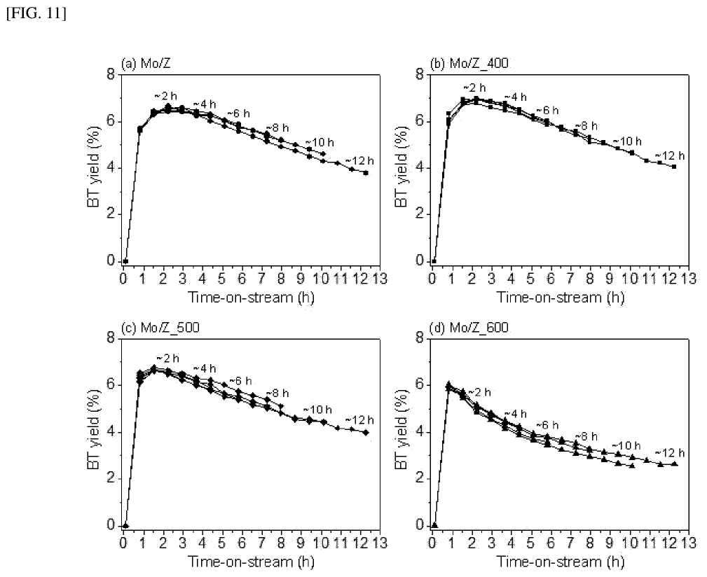

(b) of FIG. 10 shows fractions of soft coke and hard coke in the Mo-supported catalysts Mo/Z, Mo/Z_400, Mo/Z_500, Mo/Z_600 and Mo/Z_700 after the reaction obtained by decomposing the differential weight percentage profile shown in (a) of FIG. 10. The Mo-supported catalyst showed a tendency that the fraction of hard coke decreased as compared with the fraction of soft coke as the temperature of hydrothermal treatment increased. This is because BAS that promotes hard coke formation is reduced by a high temperature during the hydrothermal treatment process. In addition, since the Mo-supported catalyst is deactivated after approximately 12 hours after the reaction, it was appropriate to evaluate the coke before 12 hours. Therefore, the reaction time was divided into about 2, 4, 6, 8, 10 and 12 hours, and the MDA reaction was performed for each reaction time (TOS) to recover the used catalyst and measure TGA. The TGA measurement results of the Mo-supported catalysts (Mo/Z, Mo/Z_400, Mo/Z_500 and Mo/Z_600) were confirmed to be consistent with the BT yield (FIG. 11), which means that the evaluation of the MDA catalyst performance of all of the Mo-supported catalysts described above is reliable.

In order to evaluate the formation of coke generated during the MDA reaction, a test was performed with various reaction times, and then the TGA measurement of the catalyst was performed. That is, the amounts of soft coke and hard coke are estimated using the decomposed differential weight percentage profiles of the respective Mo-supported catalysts at different reaction times, as shown in FIGS. 12 and 13 along with the original weight changes. Thereafter, the soft coke and the hard coke were represented by graphs with respect to reaction time (h) ((c) of FIG. 10). In addition, linear regression was used to estimate the accumulation amounts for the soft coke and the hard coke, and the accumulation rates of the corresponding coke are shown next to the accumulation coke in (c) of FIG. 10, and the entire linear fit equation is summarized in Table 6.

The accumulation rates of the soft coke formed at Mo/Z., Mo/Z_400, and Mo/Z_500. were almost similar, and the Mo/Z_600 was significantly lower ((c) of FIG. 10). On the other hand, the accumulation rate of hard coke decreased monotonically in the order of Mo/Z>Mo/Z_400>Mo/Z_500>Mo/Z_600. Considering that the accumulation rate of the hard coke was the highest at Mo/Z., and the accumulation rate of the hard coke was opposite to the increase in the temperature of the hydrothermal treatment, it means that the hydrothermal treatment reduced BAS capable of promoting hard coke formation, particularly the acidic region facilitating hard coke formation, even in the TGA measurement. On the other hand, in spite of such correlation, a gradual increase in the soft coke and the hard coke according to the reaction time could not explain the rate of formation of the inactivated BT shown in (a) of FIG. 8, but it is known that the hard coke may cause MDA inactivation.

In (a) of FIG. 8, the inactivation of Mo/Z, Mo/Z_400, Mo/Z_500, and Mo/Z_600 was confirmed. At this time, since the position of the coke (inside or outside the H-ZSM-5 support) and the quantitative measurement of the coke were difficult with the TGA measurement itself, TG and Ar adsorption analysis were additionally performed. To this end, the amount of internal coke formed in the 10-MR pores (zeolite pores) related to catalytic activity was quantified at each reaction time or duration.

Specifically, the difference in the V_{10-MR} values consumed in the unreacted reaction (reaction time of 0 hours) obtained by decomposing the H-K pore size distribution and the reaction for each time (reaction times of 2, 4, 6, 8, 10 and 12 hours) was confirmed (FIGS. 14 and 15). Thereafter, the volume difference (difference in V_{10-MR} value) was multiplied by the known polycyclic coke density (d=1.22 g·cm³) to calculate the amount of internal coke for each reaction time. The amount of the external coke was determined by simply subtracting the amount of internal coke from the total amount obtained by TGA measurement. The external coke includes both a hard coke and a soft coke, and similarly, the internal coke includes both a hard coke and a soft coke.

(d) of FIG. 10 shows the amount of accumulated internal and external coke with respect to the reaction time (h). In all catalysts, the amount of the external coke continued to increase with increasing reaction time, while the amount of the internal coke increased initially and then remained constant after approximately 8 hours. Therefore, it could be confirmed that the continuous lamination of coke on the outer surface of the catalyst was the main cause of deactivation of all catalysts.

After the reaction, external coke in the catalyst blocked the pore inlets to prevent access to BAS present inside the micropores. In connection with the deactivation of the catalyst, in order to identify other causes besides blocking of the pore inlets by the external coke, the catalyst was recovered after each reaction time (about 2, 4, 6, 8, 10, and 12 hours), and the amount of BAS in the catalyst was tracked and quantified using the NH₃-TPD technique.

(a) of FIG. 16 shows the total amount of BAS_{M+S} based on NH₃-TPD of the Mo-supported catalyst after the reaction. In (c) and (d) of FIG. 10, hard coke was continuously produced according to the reaction time, and the total amount of BAS_{M+S} accessible to NH₃ decreased as the reaction time increased.

In particular, the position of the coke was confirmed through TG and Ar adsorption analysis. Referring to (d) of FIG. 10, it was confirmed that a gradual increase of the external coke, rather than the internal coke, was consistent with the tendency of BAS_{M+S}, and that the actual amount of the external coke was in inverse proportion to the amount of BAS_{M+S} shown in (a) of FIG. 6.

It was confirmed that Ar was still accessible in the 10-MR pores even though the external coke was laminated when the reaction time (TOS) was about 8 hours or more (FIGS. 14 and 15). In this case, even when the reaction time is 8 hours or more, the 10-MR pores are still preserved, and the reactants and intermediate products may approach the 10-MR pores to be further transformed, thereby continuing the formation of BT. Therefore, it could be confirmed that pore blockage by external coke was not the only cause of catalyst deactivation.

The BT formation rate (see (4a) of FIG. 8) was used to correct the total accessible BAS_{M+S} for the same reaction time, and the results are shown in (b) of FIG. 16. In (b) of FIG. 16, all catalysts exhibited similar BT formation rates at the initial reaction time, and the BT formation rates tended to decrease as the reaction time increased. This is affected by the reduction in the total BAS_{M+S} required for aromatization in the zeolite pores. The internal coke also limits the access of methane to active BAS_{M+S} together with the external coke, so that the rate of BT formation decreases as the reaction time increases, but the decrease in total BAS_{M+S} did not coincide with the decrease in 10-MR pores ((a) and (c) of FIG. 16). In order to confirm this, the BT formation rate was corrected similarly to the method of correcting the total accessible BAS_{M+S} using the 10-MR pore volume ((c) of FIG. 16) of the used catalyst ((d) of FIG. 16). Referring to (d) of FIG. 16, it could be confirmed that the trend of the corrected BT formation rate is similar to the trend of the original BT formation rate shown in (a) of FIG. 8, and is different from the trend shown in (b) of FIG. 16.

The present experiment confirmed that only the amount of 10-MR pores representing physical properties does not affect the catalyst deactivation in the MDA process. That is, although the roles of the external coke and the BAS could not be completely distinguished from each other, it could be confirmed that both the gradual increase of the external coke and the decrease in the amount of BAS_{M+S} (representative of the chemical characteristics) had an effect on the deactivation of the catalyst.

### 4. Clarification of the Effect of Hydrothermal Treatment on the Performance of MDA Catalysts

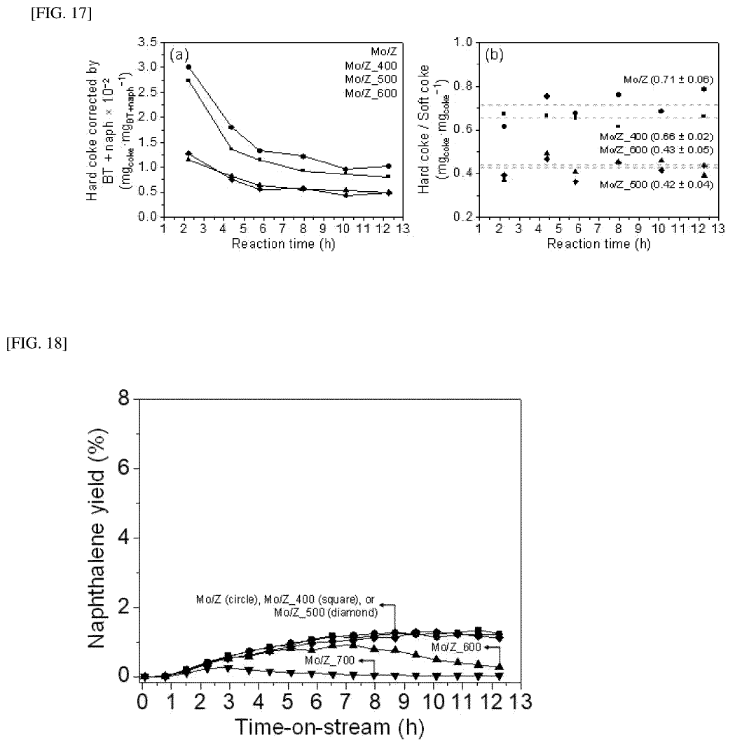

FIG. 17 is a graph showing the corrected hard coke amount of the Mo-supported catalyst and the ratio of the soft coke ratio to the hard coke according to the reaction time. In FIG. 17, (a) is the corrected hard coke amount of the Mo-supported catalyst (Mo_Z and Mo/Z_x; x=400, 500, 600) according to the reaction time, and (b) is the ratio of hard coke to soft coke. In (a) of FIG. 17, the amount of hard coke obtained from the TGA measurement in FIGS. 12 and 13 was corrected by the accumulated amount of BT and naphthalene produced according to each reaction time. In (b) of FIG. 17, the dotted line represents an average of hard coke to soft coke ratios of each catalyst. Here, the average ratio is shown along with the standard deviation. FIG. 18 is a graph showing the naphthalene yield of the Mo-supported catalyst according to the reaction time. In FIG. 18, Mo/Z (●), Mo/Z_400 (▪), Mo/Z_500 (♦), Mo/Z_600 (▴) and Mo/Z_700 (▾) are indicated, respectively.

In order to further confirm the effect of hydrothermal treatment of the Mo-supported catalyst on MDA performance, the amount of hard coke obtained by TGA measurement was corrected by the sum of the BT (FIG. 7(f))+naph (naphthalene, FIG. 18) yield accumulated during the reaction for 12 hours. This correction is due to the fact that the hard coke was mainly produced by the polymerization of aromatic products. A value obtained by the correction represents the amount of hard coke corrected to BT+naph ((a) of FIG. 17). At all reaction times (TOS), Mo/Z showed the highest amount of corrected hard coke.

At Mo/Z_400, the amount of corrected hard coke decreased, which means that the hydrothermal treatment at 400° C. reduced the BAS that promoted hard coke formation. In addition, when the temperature of the hydrothermal treatment was increased to 500° C. or 600° C., the amount of hard coke corrected at Mo/Z_500. and Mo/Z_600. was almost similar at all reaction times.

In addition, in the Mo-supported catalyst after the reaction was completed, the ratio of hard coke to soft coke did not change significantly over time ((b) of FIG. 17), which means that the composition of the coke fraction does not change due to the chemical change of the H-ZSM-5 support. The average ratio of hard coke to soft coke varied only when hydrothermal treatment was in progress, which clearly showed a specific effect on the change in chemical properties (mainly BAS) and the reduction of hard coke fraction. The decreasing order of the average ratio was Mo/Z>Mo/Z_400>Mo/Z_500˜Mo/Z_600 ((b) of FIG. 17), which was consistent with the positive trend of the corrected hard coke.

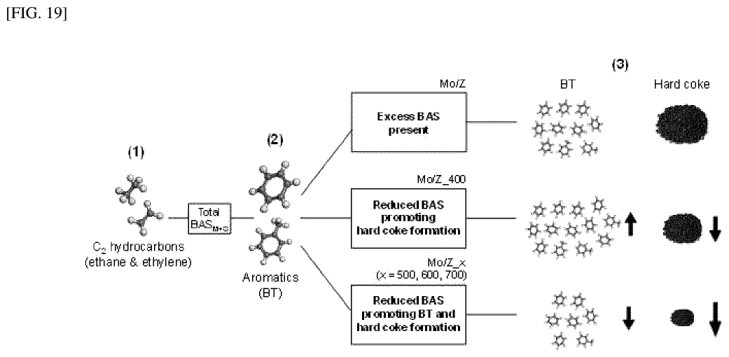

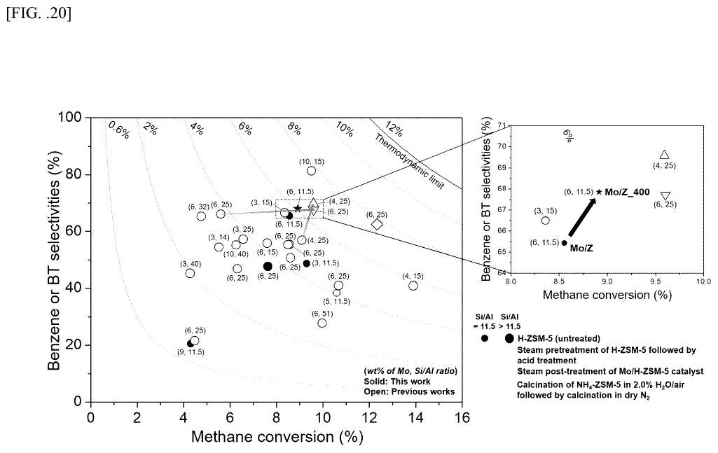

FIG. 19 is a schematic diagram of methane dehydrogenation of a Mo-supported catalyst. FIG. 19 shows the methane dehydrogenation reaction divided into step (1), step (2), and step (3) for the Mo-supported catalyst (Mo/Z and Mo/Z_x; x=400, 500, 600, 700). In step (1), starting from the C2 hydrocarbons produced by the dehydrogenation of CH4 by the active Mo species, in step (2), the intermediate hydrocarbons are aromatized by the total BAS_{M+S} in the catalyst to produce BT. In step (3), it is possible to confirm the difference between the accumulated BT yield and the hard coke, and the resulting MDA activity and stability according to the reacted catalyst. The C2 hydrocarbons are aromatized by the BAS present in the conformational selective pores of the zeolite support to produce the desired BT product. After that, various BT generation properties start to appear depending on the catalyst used in the reaction.

In this example, Mo/Z_400 showed the best MDA performance, which is believed to be due to the reduction in BAS that promotes hard coke production. The performance of Mo/Z follows this performance, with Mo/Z having an excess of BAS that further converts the desired product (BT) into coke. In addition, the MDA performance of Mo/Z_500 was different from that of Mo/Z_600 and Mo/Z_700 in terms of CH4 conversion and BT production, but Mo/Z_500, Mo/Z_600, and Mo/Z_700 exhibited lower MDA performance than that of Mo/Z_400 due to excessive dealumination of the zeolite support.

### 5. Comparison of Performance of MDA Catalyst of Mo-Supported Catalyst to Mo-Based ZSM-5 Zeolites Reported in Previous Studies

FIG. 20 is a result of comparing the performance of the Mo-supported H-ZSM-5 catalyst disclosed in the literature with the performance of the Mo-supported catalysts (Mo/Z, Mo/Z_400) according to the present example. FIG. 20 shows the MDA performance of the tested catalyst at a TOS of 6 to 7 hours under conditions similar to the reaction conditions (about 700° C., 1 atmosphere, 1500-1800 mL·g⁻¹·h⁻¹) (about 700° C., 1 atmosphere, 1500 mL·g⁻¹·h⁻¹).

A catalyst (H-ZSM-5 (untreated)) having various Mo contents or Si/Al ratios and not subjected to hydrothermal treatment was prepared and evaluated. Most of the catalyst (indicated by open circles) higher than the Si/Al ratio (11.5) of Mo/Z (CH₄ conversion of about 8.6% and selectivity of 65.4%) exhibited low BT or benzene yield. The highest performing catalyst among the activated untreated catalysts known in the literature (about 9.5% CH₄ conversion and 81.3% selectivity) had a higher Mo content relative to Mo/Z, but the Si/Al ratio was nearly the same.

As a result of preparing and evaluating the catalyst having the same Si/Al ratio (11.5) as the higher Mo content (9 wt %), the catalyst performance was very low compared to Mo/Z (about 4.3% CH₄ conversion and 20.5% selectivity). As a result of evaluating a catalyst having a Si/Al ratio (11.5) equal to the lower Mo content (3 wt %), the catalyst performance was relatively low compared to Mo/Z (about 9.3% CH₄ conversion and 48.7% selectivity). That is, when the catalyst was used as the MDA catalyst, it was confirmed that the highest performance was achieved by the Mo content of 6 wt % and the Si/Al ratio of 11.5.

In addition to the untreated catalyst, catalysts which were subjected to further vapor treatment (reduced amount of BAS; triangular, inverted triangular, rhombus, star-shaped symbols) were included in FIG. 20. The catalyst activity of the steam-treated catalyst was found to be slightly better than that of Mo/Z_400 (CH₄ conversion of about 8.9% and selectivity of 67.8%), but the steam-treated catalyst is more complicated and difficult to control than the method according to the present embodiment, and thus there is a problem in that mass production is difficult.

The steam-treated catalysts may be (1) a catalyst in which the H-ZSM-5 support is pretreated with steam and then subjected to acid treatment (indicated by triangles) or (2) a catalyst in which the NH4-ZSM-5 support is first calcined in humid air and then calcined in dry N2 (indicated by diamonds). Since the steam-treated catalysts had a Si/Al ratio of 25, which is nearly twice the Mo/Z_400, the corresponding MDA performance (about 7.6% CH₄ conversion and 47.7% selectivity) was not satisfactory compared to the MDA performance of the hydrothermally treated H-ZSM-5 support as a result of preparing an untreated catalyst having a Mo content of 6 wt % and a Si/Al ratio of 25 to compare them.

### 4. Conclusion

It could be confirmed that dealumination by hydrothermal treatment of the H-ZSM-5 support before Mo was supported had an effect on the performance of the MDA catalyst.

Specifically, dealumination of the zeolite support by hydrothermal treatment was performed using steam in the range of 400-700° C. Thereafter, the performance of the catalyst that was not subjected to hydrothermal treatment and the catalyst that was subjected to hydrothermal treatment was evaluated, and the difference in BT production rate was confirmed. There was a difference in the rate of BT production according to the order of increase in the activity of the catalyst, and the rate of BT production was in the order of Mo/Z_400>Mo/Z≈Mo/Z_500>Mo/Z_600>Mo/Z_700.

As a result of TGA measurement of the catalyst recovered after performing the MDA reaction for each reaction time, the production of hard coke was suppressed, and the degree of inhibition was proportional to the temperature of the hydrothermal treatment. In particular, in the case of Mo/Z_400, BAS, which is known to promote hard coke production, was reduced by hydrothermal treatment at 400° C., and a higher BT production rate was shown compared to Mo/Z, which was not subjected to hydrothermal treatment with excess BAS. On the other hand, it could be confirmed that Mo/Z_500, Mo/Z_600, and Mo/Z_700, which are catalysts having increased temperature of hydrothermal treatment, were not advantageous for further increasing the BT production rate. That is, the amount of hard coke was effectively reduced by hydrothermal treatment, but the BAS required for the production of BT, the desired product, was also greatly reduced.

The physicochemical properties of the catalyst recovered after the reaction time elapsed were confirmed. In the Mo-supported catalyst, the amount of BAS and micropore volume were considered as indicators representing chemical and physical properties, respectively. The chemical and physical properties were used to understand the catalyst inert properties in MDA in Mo-supported catalysts.

In the present example, it was confirmed that the catalyst had improved MDA performance due to the optimum amount of BAS of Mo/Z_400, and it was confirmed that the BT yield could be improved through the inhibition of coke formation, which is the final product of the catalyst, by adjusting the amount of BAS to improve the performance of the Mo-containing catalyst.

Those skilled in the art to which the present invention pertains will be able to understand that the present invention may be embodied in other specific forms without changing the technical spirit or essential features thereof. Therefore, it should be understood that the embodiments described above are exemplary and not restrictive in all aspects. The scope of the present invention is indicated by the scope of a patent claim to be described later rather than the detailed description, and it should be interpreted that all changes or modifications derived from the meaning and scope of the scope of the patent claim and the equal concept thereof are included in the scope of the present invention.

What is claimed is:

1. A method for manufacturing a dehydroaromatization catalyst, the method comprising: dealuminating a zeolite by hydrothermal treatment at a first temperature; and supporting molybdenum (Mo) in the dealuminated zeolite, wherein a Fourier-transform infrared (FT-IR) spectrum is repeatedly measured 50 to 300 times in a range of 1600-1400 cm⁻¹, and BrØnsted acid sites obtained by integrating the wavelength of a peak of 1545 cm⁻¹ are 300 μmol·g⁻¹ to 850 μmol·g⁻¹.

2. The method for manufacturing a dehydroaromatization catalyst according to claim 1, further comprising primarily calcining the zeolite at a temperature range of 300° C. to 700° C. for 10 hours to 24 hours before performing the hydrothermal treatment.

3. The method for manufacturing a dehydroaromatization catalyst according to claim 1, wherein the first temperature is 300° C. to 500° C., and the hydrothermal treatment is carried out for 6 hours to 48 hours in a tubular furnace at a flow rate of 800 mL·min⁻¹ to 1500 mL·min⁻¹ of air containing 5 mol % to 20 mol % water vapor.

4. The method for manufacturing a dehydroaromatization catalyst according to claim 1, wherein the zeolite has a Si/Al of 10 to 20.

5. The method for manufacturing a dehydroaromatization catalyst according to claim 1, wherein the content of molybdenum (Mo) is 4 wt % to 8 wt %.

6. The method for manufacturing a dehydroaromatization catalyst according to claim 1, wherein the step of supporting molybdenum (Mo) comprises impregnating the dealuminated zeolite in an aqueous solution comprising Mo for 10 minutes to 30 minutes at a temperature range of 20° C. to 30° C., drying the dealuminated zeolite at a temperature range of 50° C. to 90° C. for 8 hours to 24 hours, heating the dealuminated zeolite at 0.5° C.·min⁻¹. to 2° C.·min⁻¹. in an air atmosphere of 200 mL·min⁻¹ to 500 mL·min⁻¹ and performing a secondary calcination for 10 hours to 24 hours at a temperature range of 300° C. to 700° C.

7. The method for manufacturing a dehydroaromatization catalyst of claim 6, wherein the aqueous solution containing Mo comprises at least one of ammonium heptamolybdate tetrahydrate ((NH₄)6Mo₇O₂₄·4H₂O), ammonium phosphate hydrate ((NH₄)₃PMo₁₂O₄₀·xH₂O), phosphorus molybdate hydrate (12MoO₃·H₃PO₄·xH₂O), sodium phosphate hydrate (Na₃[P(Mo₃O₁₀)₄]·xH₂O), sodium molybdate 2hydrate (Na₂MoO₄·2H₂O), molybdenum contaminant (MoCl₅), molybdenum(VI) tetrachloride oxide (MoOCl₄), sodium molybdate (Na₂MoO₄), and molybdenum(IV) sulfide (MoS₂).

8. The method for manufacturing a dehydroaromatization catalyst according to claim 1, wherein the dehydroaromatization catalyst comprises soft coke and hard coke, and the hard coke is 25 parts by weight to 42 parts by weight based on 100 parts by weight of the soft coke and the hard coke, the soft coke is graphite, and the hard coke is polyaromatic hydrocarbons or polycyclic aromatic compounds.

9. The method for manufacturing a dehydroaromatization catalyst according to claim 1, wherein the dehydroaromatization catalyst comprises an internal coke provided inside the pores of the zeolite and an external coke present on the surface of the zeolite, and the external coke is 60 parts by weight to 75 parts by weight based on 100 parts by weight of the total of the internal coke and the external coke.

10. The method for manufacturing a dehydroaromatization catalyst according to claim 1, wherein when the first temperature increases, the amount of Bronsted acid sites decreases, and the molybdenum is present in the pores of the zeolite.

11. The method for manufacturing a dehydroaromatization catalyst according to claim 1, wherein the dehydroaromatization catalyst has an average particle size of 150 μm to 250 μm, and a methane conversion rate according to the following formula is 2.5% or more:
Methane conversion rate (%)=(number of moles of CH₄ reacted/number of moles of CH₄ supplied)×100 (Formula)

12. The method for manufacturing a dehydroaromatization catalyst according to claim 1, wherein in the X-ray diffraction (XRD) analysis spectrum using Cu—Kα, the peak corresponding to the (021) plane is not present, and the peak of (101) is shown to be stronger than the peak of (200/020) and the peak of (111).

13. A dehydroaromatization catalyst comprising a zeolite and molybdenum (Mo), wherein the dehydroaromatization catalyst is prepared according to the method of claim 1.

14. The dehydroaromatization catalyst of claim 13, wherein the zeolite is a ZSM-5-based zeolite, a content of the molybdenum (Mo) is 4 wt % to 8 wt %, and the molybdenum is present in pores of the zeolite.

15. The dehydroaromatization catalyst according to claim 13, wherein the Fourier-transform infrared (FT-IR) spectrum is repeatedly measured a total of 50 to 300 times in a range of 1600-1400 cm⁻¹, and the BrØnsted acid sites obtained by integrating the wavelength of the peak area of 1545 cm⁻¹ are 300 μmol·g⁻¹ to 850 μmol·g⁻¹.

16. The dehydroaromatization catalyst of claim 13, wherein the dehydroaromatization catalyst comprises a soft coke and a hard coke, wherein, based on 100 parts by weight of the soft coke and the hard coke, the hard coke is 25 parts by weight to 42 parts by weight, the soft coke is graphite, and the hard coke is polyaromatic hydrocarbons or polycyclic aromatic compounds.

17. The dehydroaromatization catalyst of claim 13, wherein the dehydroaromatization catalyst comprises an internal coke provided inside the pores of the zeolite and an external coke present on the surface of the zeolite, and the external coke is 60 parts by weight to 75 parts by weight based on 100 parts by weight of the total of the internal coke and the external coke.

18. The dehydroaromatization catalyst according to claim 13, wherein the dehydroaromatization catalyst has an average particle size of 150 μm to 250 μm, and a methane conversion rate according to the following formula is 2.5% or more:
Methane conversion rate (%)=(number of moles of CH₄ reacted/number of moles of CH₄ supplied)×100 (Formula)

19. The dehydroaromatization catalyst of claim 13, wherein in the X-ray diffraction (XRD) analysis spectrum using Cu—Kα, there is no peak corresponding to the (021) plane, and the peak of (101) is stronger than the peak of (200/020) and the peak of (111).

20. The dehydroaromatization catalyst of claim 13, wherein the dehydroaromatization catalyst is configured to perform a dehydroaromatization reaction with a reactant comprising methane to prepare an aromatic compound, and the aromatic compound comprises one or more of benzene, toluene, xylene, naphthalene, and coke.
